(19) United States
(12) Patent Application Publication
(10) Pub. No.: US 2026/0283585 A1
(43) Pub. Date: Sep. 24, 2026

(54) DEVICE AND METHOD FOR CAPTURING AND EVALUATING VIBROACOUSTIC SIGNALS

(71) Applicant: SURAG Medical GmbH, Leipzig (DE)

(72) Inventors: Alfredo Guillermo ILLANES MANRIQUEZ, Magdeburg (DE); Thomas SÜHN, Magdeburg (DE); Nazila ESMAEILI, Magdeburg (DE); Moritz Herbert SPILLER, Magdeburg (DE)

(73) Assignee: SURAG Medical GmbH, Leipzig (DE)

(21) Appl. No.: 19/156,461

(22) PCT Filed: Feb. 15, 2024
(86) PCT No.: PCT/EP2024/053795

§ 371 Date: Aug. 14, 2025

(30) Foreign Application Priority Data
Feb. 16, 2023 (DE) 10 2023 103 883.4

(51) Int. Cl. A61B 7/02; A61B 5/00; A61B 5/0205

(52) U.S. Cl. CPC A61B 7/023; A61B 5/0205; A61B 5/7267; A61B 2505/05; A61B 2562/0204

(57) ABSTRACT

The invention relates to a device for connection to a medical instrument for capturing vibroacoustic signals, wherein the device comprises a signal capturing device (4) with a coupling element (4a) for connecting a medical instrument (2), at least one sensor (4c) for capturing vibroacoustic signals, an analog-to-digital conversion (4e) for converting the analog signals captured by the at least one sensor into digital data, a controller (4f) and a communication unit (4g) for data exchange. The invention also relates to a computer-implemented method for evaluating the vibroacoustic signals.

The invention relates to a device and a method for capturing and evaluating vibroacoustic signals, in particular during and in connection with minimally invasive interventions.

In the conventional open surgery, sensory impressions are an important source of information for the surgeon. During an intervention, the surgeon relies on the visual, tactile and auditory senses to assess the condition of organs and tissues. For example, with the visual sense, surgeons can directly survey the entire surgical area and observe the affected areas of the patient's body; with the tactile sense, they can examine organs and tissues directly with the hand; and with the auditory sense, surgeons can hear the sounds of organs such as the heart, lungs and blood vessels.

It is known that these sensory impressions can only be perceived to a very limited extent, if at all, and only indirectly via a medical instrument, particularly during minimally invasive surgery (MIS).

MIS has become the standard for many surgical procedures. In minimally invasive procedures, special medical instruments are usually inserted into the body through small incisions. Accordingly, the instruments usually have an elongated shape with the smallest possible diameter.

The MIS has enormous advantages for the patient compared to open surgery, such as shorter recovery times and less tissue trauma, but the limited sensory information available to the surgeon during MIS can lead to a higher number of errors and therefore complications.

The MIS also places special demands on surgeons in the visual field, as visual transmission is usually only possible via cameras, which are also inserted in a minimally invasive manner.

Acoustic perception of the organs is also limited in contrast to open surgery, as the operating area is essentially closed and there is no direct access to the inside of the patient's body.

Furthermore, the sense of touch is virtually eliminated as the surgeons cannot insert their fingers into the patient's body, but operate special minimally invasive instruments using more or less rigid control elements. Accordingly, surgeons cannot rely on the skills and experience they have acquired during conventional operations to assess and evaluate the condition of the patient, organs or tissue.

Various assistive technologies have been developed to support surgeons during minimally invasive procedures and at least partially replace the lack of sensors. These technologies are intended, for example, to help with the guidance of surgical instruments and the recognition of anatomical structures, as well as to enable an overall improved perception of surgical conditions.

All known solutions require physical interaction of the instrument with the tissue and differ technically mainly in the location and type of sensors used to measure, for example, force, torque, pressure, impedance and vibration.

Direct palpation generally uses an instrument tip fitted with sensors, a special probe or an arrangement of transducers in direct contact with the target structure. Disadvantages include the design and manufacturing challenges of integrating sensors on the instrument itself without compromising the original functionality or quality of the instrument, as well as compliance with sterilization and biocompatibility requirements.

Indirect measurements, on the other hand, are characterized by the fact that a single transducer is located outside the body but close to the actuating mechanism of the instrument. This alleviates some of the limitations of direct sensing, but spatial resolution is limited to point assessment and measurement accuracy and measurement consistency is limited.

Known visual technologies are designed to support the surgeon's visual perception, sensor-based solutions have also been developed to support the surgeon's sense of touch, for example, and the presentation of information in the form of acoustic feedback has been introduced to support the surgeon's sense of hearing.

In MIS, for example, visual support is currently provided by RGB cameras during laparoscopic or robot-assisted procedures by inserting an endoscopic camera through a tiny incision in the surgical area.

This supporting information is displayed on a monitor in the form of real-time videos having high-quality and high-resolution and can also be combined with depth information to provide a three-dimensional representation of the surgical site.

Meanwhile, the post-processing and analysis of the resulting videos is part of a new medical technology research field called Surgical Data Science (SDS), which can involve various areas such as surgical decision support, surgical training and contextual support such as monitoring interventions, predicting the remaining operation time, monitoring interventions to facilitate scheduling, monitoring interventions to predict resource requirements, recognizing surgical phases, incorporating collaborative robots, predicting poor outcomes, warning of potential problems and retrospectively identifying the causes of poor outcomes.

The main disadvantage of video storage and video analysis is the large amount of storage space for video recordings and the limited dynamic range that video can provide. Due to the low frame rate of video, the information that can be obtained from this type of data is usually limited to that which can be perceived by humans. However, it is known that in the case of biological and mechanical processes, the data obtained can also provide insights into areas that are difficult or impossible to perceive with the human senses.

Another disadvantage of endoscopic videos in particular is that the surgical procedure is usually only recorded from one perspective. In laparoscopic surgery, only a single endoscopic camera is placed in the body, which limits the monitoring of the surgical procedure to this single perspective.

In addition to RGB cameras, surgeons are supported in some types of minimally invasive procedures by other imaging techniques such as magnetic resonance imaging, X-ray, computer tomography and ultrasound.

Depending on the procedure used, the surgeon's visual perception can also be enhanced by combining these imaging technologies with instrument tracking and navigation systems such as optical tracking and electromagnetic tracking to emphasize visual feedback on the position of the instrument and the targeted organ or anatomical structure.

In fact, the majority of current developments that attempt to compensate for the surgeon's diminished sensory perception in MIS are limited to improving visual perception.

However, displaying information on a screen is often not an ideal solution, as surgeons have to take their eyes off the operating area to look at the corresponding monitor and receive the information provided there. The three-dimensional display of information intended as an additional aid is also not always intuitive and usually requires prior training and experience in order to be interpreted correctly.

In addition, the real-time videos provided during the operation do not have an audio track and therefore do not provide any additional acoustic information that could further clarify the course of the operation. Nevertheless, they require a lot of storage space for possible post-evaluation and are computationally intensive to process.

Last but not least, video recordings have the major disadvantage that they only ever have a very limited field of view (Blickfeld; FoV) and—unlike other imaging methods—cannot look beneath the surface of structures. The line of sight (Sichtlinie; LoS) of the video and thus that of the surgeon ends at the first opaque surface. In both cases (FoV, LoS), interactions outside the image areas cannot be recognized by the surgeon using the known video procedures and cannot be recorded by the system and therefore cannot be evaluated.

Accordingly, there is a great need for further sensor technologies in order to provide additional data and information that cannot be provided by video systems in particular.

Various sensor-based developments are known in the state of the art as a supplement or alternative to video surveillance, specifically to replace the surgeon's lack of touch during minimally invasive procedures. These solutions use various types of sensor technology to measure force, torque, pressure, impedance, vibrations or to record any kind of tactile information. Different types of probes to measure tissue characteristics have also been proposed.

Wider acceptance of one of these systems in the clinical field is not yet apparent, nor is it foreseeable in the future. This is because such systems are often extremely cost-intensive and cannot be combined with known and available standard instruments.

In most systems, namely the necessary sensors are embedded at the distal end of the instrument, which requires the development of proprietary instruments that have to be adapted to the respective system. With such instruments, the sensors must also be inserted into the patient's body. Additional cabling and active components are also usually required, which must also be embedded in the instrument.

In particular, auditory support for minimally invasive procedures is still insufficiently researched. Nevertheless, some attempts have been made to develop technologies to support information presentation and enhance hearing in MIS, focusing on two areas: auditory presentation and sonification.

The auditory visualization is mainly used in combination with image-guided interventional systems in a variety of minimally invasive procedures, such as needle placement (among others as part of a radiofrequency needle ablation or a needle biopsy), drilling of the temporal bone, volumetric tissue resection or telerobotic suture tightening.

For this purpose, data from various sources—including, for example, the distance information provided by the navigation system used during the intervention—is mapped as changing parameters of a sound generator, which generates corresponding acoustic feedback. Auditory display methods include, for example:
1) Warning messages, where sounds are played when the captured values reach, exceed or fall below a predetermined threshold;
2) auditory symbols, which are familiar sounds used to convey information about events by analogy to everyday sounds; and
3) parameter mappings that translate continuous changes in a data set into continuous changes in audio parameters, such as pitch or loudness.

Sonification focuses on the use of non-speech audio data to acoustically associate the key physical parameters of a sound, such as frequency (pitch), duration and intensity, with an important property of the signal being represented, such as timbre, rhythm and harmony. Several research studies on sonification are taking place in the fields of heart rate analysis, biorhythm analysis, electrocardiogramolecular sequences, cellular kinetics, medical image diagnosis and electroencephalography.

Although these well-known methods can provide the surgeon with a better perception during a minimally invasive procedure, these methods are less researched and less developed for minimally invasive procedures. So far, they cannot or at least not completely compensate for the missing part of the sense of hearing in MIS.

It is therefore the task of the invention to provide a device and a method which do not have the disadvantages mentioned.

In particular, it is a task of the invention to provide a device and a method which are suitable, in particular during or in connection with minimally invasive interventions, for compensating for the missing sensory impressions by means of suitable feedback based on the detection and evaluation of vibroacoustic signals.

It is further a task of the invention, based on the inventive device and the inventive method, to provide a surgical monitoring system that is cost-efficient and easy to use. The system should also offer a large dynamic range that goes beyond human perception and can therefore provide a wide range of possibilities for observing a process.

This task is solved by a device with the features of claim 1 and a computer-implemented method with the features of claim 4. Advantageous embodiments are each the subject of the dependent claims. It should be noted that the features listed individually in the claims can also be combined with one another in any technologically useful manner and thus demonstrate further embodiments of the invention.

Accordingly, the present invention relates to a device and a computer-implemented method for capturing and evaluating vibroacoustic signals. The device is suitable for use with medical instruments.

The method according to the invention relates, for example, to the processing of vibroacoustic signals for audification and augmentation as well as for evaluation, monitoring and documentation of medical procedures or medical tasks. Medical procedures may include, for example, laparoscopic and robot-assisted surgery, arthroscopy or needle interventions, medical tasks may include, for example, palpation or ablation, to name just a few examples in this non-exhaustive list.

In a broader sense, “audification” (in German: Audifikation) refers to the translation of a data set into an acoustically perceptible event. The origin of the data set can be different and does not necessarily have to relate to an event that can be perceived by our human senses. The origin of the data set can therefore be in the inaudible frequency range or below the human perception threshold for vibrations, for example. The original data set can initially also be completely detached from an audible event; in principle, any data sets of any events can be made audible and thus experienced with audification.

The audification is achieved by connecting one or more devices according to the invention to one or more medical instruments. The terms “medical instrument” or “instrument” are intended here to summarize all medical, surgical or interventional tools and instruments that are used by the persons performing a medical procedure, for example a non-invasive intervention or a conventional open operation or a minimally invasive intervention, in each case also in the context of diagnostics.

The device according to the invention comprises a signal acquisition device, a signal processing/data processing device and optionally an output device.

The device according to the invention can preferably be connected to a medical instrument as an add-on device. For this purpose, a coupling element is provided on the signal acquisition device, which is preferably adapted to known standard connections, such as Luer-Lock connections. However, the coupling element can also be provided universally for different connections.

Alternatively, the device according to the invention can also be implemented in the respective medical instrument. This is particularly advantageous if the device is used in robot-assisted surgery (RAS), for example, and is part of the robot module.

The coupling element can comprise a positive-locking opening for inserting a medical instrument or a device which may have a force-locking effect, for example in the form of a clamp element, with which the device according to the invention can be attached to a medical instrument.

Optionally, the device according to the invention comprises an adapter element which is intended to produce a precisely fitting, i.e. a positive, frictional and/or non-positive connection between the medical instrument and the coupling element, if such further adaptation is necessary here.

Coupling element and adapter element are intended to transmit vibroacoustic signals from the medical instrument to a sensor with as little interference as possible.

When a part of the medical instrument, for example the shaft or initially the tip, comes into contact with a tissue as defined below, a mechanical vibration is generated at the contact point. The vibration propagates passively, without the need for an active or activating component, as a vibroacoustic wave through the entire instrument and thus to any point on the physical surface of the medical instrument. This includes propagation to an object connected to the medical instrument such as other instruments or propagation to another structure.

By attaching a measuring system in the form of the device according to the invention, it is accordingly possible to measure the vibration propagating on the instrument, i.e. the vibroacoustic signals, as a physical quantity and to carry out a quantitative and qualitative evaluation.

The measured quantity is directly related to the interaction of the instrument with the tissue or the use of the instrument itself, for example during the interaction of the two cutting edges of laparoscopic scissors or the mere picking up or putting down of an instrument before or after use.

If several instruments are used during an intervention, which is the rule in most medical interventions, a device according to the invention can be connected to each instrument in order to measure vibroacoustic signals resulting from the interaction between the instrument and the environment and in particular between the instrument and the tissue.

An “intervention” should be understood to mean both operations in the sense of classical open surgery and minimally invasive operations. Even if the present invention is particularly oriented towards the requirements of minimally invasive procedures, the application of the invention can be advantageous for procedures in any medical environment.

Vibroacoustic signals, hereinafter also referred to as “VA signals”, are to be understood as signals which are based on a vibration of the medical instrument, whereby the vibration and thus the vibroacoustic signal is the result of the interaction of the instrument with its environment, i.e. in particular with the respective surrounding tissue.

In particular, in the preferred embodiment of the invention, no active excitation of the medical instrument is provided, although this would be conceivable in alternative embodiments.

The term “tissue” is to be understood broadly and includes all natural, biological or artificial physical structures, surfaces, tissues or objects that can interact with the instrument.

Accordingly, the device according to the invention further comprises, as part of the signal detection device, at least one sensor for capturing vibroacoustic signals, in particular an acoustic or vibration sensor, i.e. a sound-converting sensor, such as a microphone.

Accordingly, the coupling element is connected to the instrument as well as directly or indirectly to the at least one sensor in a sound-transmitting manner and is suitable for transmitting vibroacoustic signals from the instrument to the at least one sensor with as little interference and loss as possible.

In the case of a direct connection, the at least one sensor is connected directly to the coupling element, i.e. in particular by force, form-fit and/or frictional connection, whereby a piezo or crystal microphone is preferably used as the sensor in this case. The use of other sound-converting sensors is conceivable.

In an indirect connection, the coupling element is not connected directly to a sensor, but preferably not permanently but at least non-positively or frictionally connected, i.e. effect-transmitting, to a membrane that transmits the vibroacoustic signals into a sound chamber adjacent to the membrane.

Such an arrangement with a membrane and a sound chamber is also referred to below as a mechanical amplification mechanism.

Accordingly, the vibroacoustic signals have an impulse component which, usually via the coupling element, acts in an axial direction, i.e. perpendicular to the membrane, and which leads to excitation or deflection of the membrane.

The vibroacoustic signals have an additional bending moment at the coupling point of the coupling element with the membrane, whereby both the axial impulse component and the bending moment generate a deflection of the membrane and thus a sound wave or pressure wave in the sound chamber adjacent to the membrane.

The instrument itself can act as a lever arm for the resulting moment and amplify the deflection of the membrane accordingly.

In this case, the coupling element is therefore suitable for transmitting vibroacoustic signals from the instrument to the membrane with as little interference and loss as possible.

The excitation or deflection of the membrane therefore generates a sound wave or pressure wave in the sound chamber adjacent to the membrane.

The sound chamber is provided as a cavity and is preferably designed in such a way that it tapers from the membrane towards the sensor. Accordingly, the sensor picks up the vibroacoustic signals from the sound chamber as sound waves amplified by the membrane. The membrane is accordingly suitable for amplifying the vibroacoustic signals, whereby the sound chamber leads to an additional amplification of the signal.

In principle, it is also conceivable that the vibroacoustic signals are transmitted directly from the coupling element into the sound chamber, i.e. without an intermediate membrane.

The sound chamber can optionally be filled with a gaseous or liquid fluid in order to create the desired transmission properties.

The at least one sensor is provided on the sound chamber. Preferably, the sound chamber is delimited by the sensor, but embodiments in which the sensor is provided inside the sound chamber are also conceivable. Embodiments are also conceivable in which the sensor is provided outside the sound chamber but preferably connected to it.

For the indirect connection described, an air-bound microphone with a second membrane as a transducer element is preferably provided.

The second membrane of the sensor, for example that of the air-bound microphone, preferably has a significantly smaller diameter than the membrane of the sound chamber. In combination with the sound chamber, which preferably tapers towards the sensor, the different diameters of the membranes lead to a significant amplification of the deflection of the second membrane.

The arrangement leads to a considerably increased sensitivity of the device even to minimal structural vibrations of the instrument resulting from interaction forces on the instrument due to the amplification caused by the membrane combination and, if necessary, due to the leverage effect of the instrument.

In addition to these sensors, which are suitable for capturing vibroacoustic signals, further sensors can be provided on the signal acquisition device, in particular acceleration sensors, gyroscopes, force sensors, friction sensors, vibration sensors or pressure sensors. The implementation of optical sensors is also conceivable, in particular interferometers for vibration measurement. The various sensors can be used to record and process different types of optical and mechanical measured variables.

The vibroacoustic signals recorded by the at least one sensor are optionally further processed at the analog stage, i.e. they are optionally fed to an analog signal pre-processing stage. The further or pre-processing at the analog stage can in particular include filtering and amplification of the analog signal.

The data can then be digitized and transmitted to the data processing/signal processing device.

For this purpose, the device according to the invention comprises an analog-to-digital converter that converts the analog signals into digital data. Such a converter can also be part of the at least one sensor.

The signal acquisition device comprises a communication unit for transmitting or exchanging the digitized data.

The signals captured by the sensor and possibly processed in analog form are transmitted in digitized form as a digital signal or as digital data to the data processing/signal processing device, which processes the signal or data further. The data processing/signal processing device can be embedded in the device according to the invention together with the signal acquisition device or connected to it by wire or wirelessly.

The data processing/signal processing device is accordingly provided to execute the computer-implemented method for processing the digitized vibroacoustic signals and to provide the result of the processing and, for example, to pass it on to the output device in order to trigger a corresponding desired output.

In other words, the data processing/signal processing device comprises a non-transitory computer-readable medium containing instructions which, when executed by at least one processor, cause the at least one processor to perform operations enabling vibroacoustic signals to be processed and the result to be provided for transmission to the output device or directly forwarded to cause the output device to store or output a corresponding feedback to the user.

The output device is accordingly intended to output the result of the data processing/signal processing device or to store it for later use. The output can take the form of an optical, acoustic or haptic signal, for example, or a combination of one or more of these signals.

The signal acquisition device and the data processing/signal processing device represent, in addition to the computer-implemented method, the essential technical elements of the device according to the invention. These elements can be arranged and grouped in different ways.

For example, the signal acquisition device and the data processing/signal processing device may be provided in one housing.

In specific embodiments, the optional output device may also be part of the single-housing embodiment.

However, embodiments of the device according to the invention in which the device comprises several individual elements are also conceivable. In this context, “individual” means that these individual elements are not arranged in a single housing, but that at least one of the elements is connected to the other elements only via a wire or wirelessly.

For example, embodiments are conceivable in which a wired or wireless transmission of the digitized data from the signal acquisition device to a spatially separated data processing/signal processing device takes place.

Similarly, embodiments are conceivable in which, for example, only the signal acquisition device and an output device are arranged in one housing, so that the user receives the feedback via the output device directly at the point of acquisition, but the data processing can take place spatially separated in the data processing/signal processing device.

The output device can be part of the device according to the invention, but the result of the data processing/signal processing device can also be output via an external output device that is not part of the device according to the invention. Possible output devices can be computers, smartphones, laptops, tablets or other devices that can convert the corresponding input of the data processing/signal processing device into a desired output.

In the case of the RAS, it is conceivable that the result of the data processing is used directly to control the RAS system and thus for further execution of the operation, i.e. in this sense there is no output in the form of a signal, but further processing of the result of the data processing/signal processing device takes place.

Accordingly, both the output of data and the nature of the output device are to be understood broadly in the sense of the present invention, in that the output does not necessarily have to be in acoustic, haptic or visual form, for example, but the output can also be used for further control or planning of an operation or the movement of a robot, for example.

Accordingly, the output device can also comprise a storage or data forwarding function.

The device according to the invention can be connected directly or indirectly to the instrument to be measured or audified.

A direct connection results in a direct measurement. A direct measurement means that the device is connected directly to the instrument to be audified. An example of this is the audification of a biopsy needle, a palpation rod, a palpation hook or a probe.

An indirect connection leads to an indirect measurement. An indirect measurement means that a first instrument to be audified is measured by measuring a second instrument to which the first instrument is connected in a sound-transmitting or vibration-transmitting manner.

An example of this is the audification of a laparoscopic grasper, in which the device according to the invention is not connected directly to the laparoscopic grasper, but to the trocar into which the grasper is inserted.

The vibroacoustic signals of interest are therefore not measured directly at the laparoscopic grasper, but indirectly at the trocar to which these vibroacoustic signals are transmitted by the laparoscopic grasper.

Examples of interventions that can be audified depending on the type of MIS include laparoscopic and robot-assisted surgery (RAS), such as palpation, grasping, cutting, suturing, knot/stitch tightening, suture pull-out/fraying, ablation, RF coagulation, pulsatility, collision between tools, tool change/placement of a tool through the trocar port, or collision of a tool with a specific tissue outside the field of view of the endoscope.

Another category of interventions concerns needle interventions such as needle puncture, needle-tissue-tissue passage (including puncture of an organ boundary, puncture of a tumor boundary, reaching an empty or fluid cavity, perforation or puncture of a vessel, biopsy sampling).

Another category of interventions concerns procedures with guidewires/catheters such as perforation of the vessel/arterial wall, wall friction, blockage, pulsation/flow, tool operation (among others milling, cutting, etc.), stent deployment, stent-vessel wall contact, balloon inflation or balloon-vessel wall contact.

Another category concerns arthroscopy with palpation, tapping, scraping, milling, screwing, drilling, cutting or smoothing.

### GENERAL PROCESSING METHOD

The invention also relates to a computer-implemented method for processing the captured vibroacoustic signals.

The VA signal resulting from the interaction between a medical instrument and tissue has characteristics that make it very difficult to process with standardized signal processing algorithms normally used in speech/audio processing. Characteristics of the signal that make the analysis complex include:

The non-stationarity: The statistical and spectral characteristics of a VA signal resulting from the displayed interactions between medical instrument and tissue can change abruptly within milliseconds. This leads to a range of different dynamics that can go from transient to oscillatory and then back to transient in a single interaction event within a very short time. Processing transients with known signal processing algorithms for speech and sound is very difficult, as is processing very discontinuous and highly non-stationary signals.

Many things that happen in an interaction event lead to so-called micro-noises, i.e. noises that can last less than 200 ms and which the human ear cannot perceive. For example, the puncture of a tissue-to-tissue passage during the insertion of the Veres needle lasts only about 50 ms.

The VA signal can be modulated in a variety of ways during a medical procedure. These modulations are usually non-linear dynamics that are difficult to process using standard audio or speech signal processing algorithms.

An additional complexity lies in the different energy levels that can be contained in a single interaction event. For example, when a tissue is palpated, the energy generated at the moment of first contact with the tissue can be more than ten times the energy of the effective or actual palpation. In the example of the Veres needle, the internal mechanical interaction of the needle through the triggered spring mechanism can have an energy 50 times higher than the friction generated during use or the puncture itself. The processing of signals with such a variance of energies is also complex and requires the implementation of new, inventive processes.

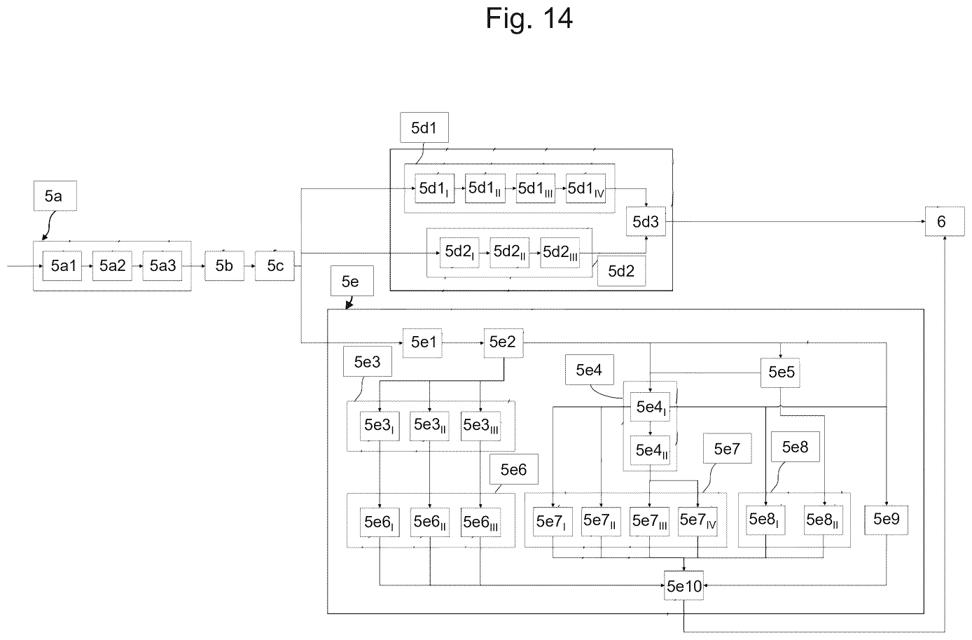

The computer-implemented process according to the invention is illustrated below in FIG. 14 and summarized here in more general terms. The exact process sequence can be seen in FIG. 14.

In a first step, the computer-implemented method according to the invention basically comprises the provision of existing digitized vibroacoustic signals.

The basic process sequence of the computer-implemented method according to the invention comprises the following steps:
(0) Providing an existing digitized vibroacoustic signal;
(I) Signal pre-processing of the vibroacoustic signal; and
(II) Identification of segments/regions of interest; and
(III) Audification; and/or
(IV) Classification of the segments/regions of interest.

Optionally, in a further step (V), for example, the output, further processing, storage, etc. of the classified segments/regions takes place.

The method includes pre-processing of the digitized VA signals. This step is intended to attenuate the various interfering noises in the VA signal and amplify the interaction events of the instrument tip or shaft.

First, the continuous components (DC) are removed from the VA signal. Then a step is taken to reduce the background noise. This can be done using spectral gating methods, for example. Finally, a signal enhancement step is performed to improve the dynamics and frequency components related to the interactions. This can be done with linear filters, adaptive filters or wavelet-based filters.

In wavelet-based filters, the discrete wavelet transform (DWT) can be used to decompose the signal into different scales and then reconstruct the signal based on the scales where high energy interactions occur.

In a next step after pre-processing, the identification of segments/regions of interest can be performed. This step can be used to segment regions of interest in the signal where important interaction events between medical instrument and tissue/surface occur. This can be used to perform a test or classification focusing only on the relevant interaction information. For this purpose, the onset and shift of a possible interaction event can be determined using, for example, abrupt change detection algorithms (e.g. Hinkley Page hypothesis tests) or with algorithms for detecting derivations.

The data processed in this way can be transferred for audification and/or for classification. For some applications, audification alone can be provided as feedback and for some other applications only classification is necessary. There are also applications that require an integration of classification and audification of interaction events.

The audification can be divided into different types. Direct audification is a direct conversion of the VA signal into an audible sound domain using various standard processing tools such as filtering, gain operators or effect generation. It can include four main sub-steps, namely filtering the signal to enhance the frequency components of signal events or signal segments of interest related to the instrument tip/shaft and tissue/surface interactions significant to a particular process. This filtering stage can be performed using various techniques, such as linear filtering, adaptive filtering, or filtering generated using any type of signal decomposition technique, such as continuous wavelet transform (CWT), discrete wavelet transform (DWT), empirical mode decomposition (EMD), or autoregressive modeling.

The signal is decomposed into different scales, modes or dynamics (depending on the technique used) and then reconstructed using scales/modes/dynamics where interaction events of interest are present, these can be for example high energy interaction events.

The volume adjustment consists of applying a gain to the VA signal to increase the volume. It can also be used to limit excessively loud events or saturated audio signal segments.

The dynamic processing is used to change the dynamic properties of the audio signal. It allows to set a ratio between the input levels and the output levels. This can be done in a single frequency band or in several frequency bands for independent, parallel dynamic processing.

The purpose of creating effects is to manipulate the sound of the audio signal. Common effects that can be used here are reverberation, reverb, convolution, echo, chorus, time stretching, transposition and harmonizer.

The indirect audification involves linking features of the VA signal with auditory parameters for the purpose of data representation. This step involves the extraction of features, which are then converted into an auditory representation by mapping the features to different parameters of the displayed sound. It comprises three main sub-steps, namely first the

Feature extraction, where different features or attributes are extracted from the signal. Different types of features can be extracted from the signal segment, such as features in the time domain, in the frequency domain or in the time-frequency domain. Examples of features in the time domain are signal strengths or signal energies, statistical features such as signal mean or signal variance and morphological features. For frequency domain features, estimates of the signal spectrum can be performed using various spectral estimation methods such as FFT, AR modeling and other methods. For features in the time/frequency domain, methods such as short-time Fourier transform (STFT), Mel frequency cepstrum, Wigner-Ville distribution, continuous wavelet transform (CWT), discrete wavelet transform (DWT), empirical mode decomposition (EMD), time-varying autoregressive modeling, cyclostationarity features, and other methods can be used.

Mapping then converts the features into audio parameters that are used to generate an auditory representation. This can be done through direct mapping of the extracted features via parameter mapping sonification, where, for example, changes in signal strength can be mapped to changes in pitch of the resulting acoustic representation; through a dynamic model that describes the temporal evolution of a system excited by the features, where the model is intended to simulate a physical sound source; or using decision rules, clustering methods, or a trained machine learning model that takes the features as inputs and produces classified events as outputs, which are then used to parameterize the sound.

The sound synthesis is used to generate specific sound characteristics.

In the sound projection step, everything necessary for transmitting the data and displaying the sound output to the feedback/storage unit is provided via one or more loudspeakers or headphones.

The classification can include several of the following steps.

First of all, the identification of transient and stationary dynamics: The VA signals resulting from the interactions between the medical instrument and tissue or surface exhibit very complex dynamics. A single interaction event can exhibit a mixture of transient and steady-state dynamics in a very short time interval. For example, a palpation event may have an extremely short transient response at the moment the tip of the instrument comes into contact with the VA excitation, followed by a steady-state response resulting from the effective palpation process. Inhomogeneities of the surface can lead to collisions of the tip with the surface, which in turn leads to a mixture of transient and steady-state dynamics. The moment the palpation stops, the contact stop can also generate transient dynamics, so that a series of transient-stationary dynamics can occur in a short time interval. This complexity means that the signal cannot be processed using standard or classical signal processing algorithms normally used for speech or musical tones, whose acoustic responses can be quite stable in a short to medium time interval.

In spectral band decomposition, the VA signal is decomposed into several narrowband signals by splitting the overall frequency band of the signal into several sub-frequency bands. The main goal is to later process different aspects of the signal separately. We assume that a signal characteristic is composed of several dynamics that represent irregularity properties of the texture such as smoothness or roughness. Therefore, the signals can be decomposed into several dynamics that can represent, for example, the levels of irregularities in the interaction. The number of frequency bands into which the signal is decomposed depends on whether the signal is generated by insertion, palpation or some other type of interaction between the instrument and the tissue/surface. The number of bands can vary between 2 and 10 frequency sub-bands. Decomposition can be performed using different methods, e.g. filter banks, continuous wavelet transform (CWT), discrete wavelet transform (DWT), pole tracking and time-varying autoregressive modeling, empirical mode decomposition (EMD), etc.

Both the previously described identification of transient and stationary dynamics as well as the spectral band decomposition and their combination are new and inventive processing methods that are not known in the literature. As mentioned above, the VA signal contains many different dynamics in the signal, which are very difficult to analyze or process together. It is necessary to reduce the complexity of the VA signal before the phase of extracting indicators and features begins. When signals are separated into different aspects, such as identifying transient and stationary dynamics into transient and oscillatory features and/or spectral band decomposition into different bandwidths and/or demodulation of the signal in different modes, features can be better associated with the extracted aspects.

Since interactions can be highly associated with abrupt changes (contact with tissue, tissue clasping, puncture and so on), abrupt change detectors should be used to assess these changes in order to account for the full extent of these changes.

Calculation of indicators: Before the feature extraction step, it is necessary to first extract indicators from the signals. This indicator calculation usually receives one or more signals as input and provides one or more signals or matrices as output. In the present signal processing methodology, three types of indicators are used, the first being time domain indicators, i.e. indicators that are calculated directly from the time series.

Three types of time domain indicators can be calculated, namely statistical indicators: Indicators that are calculated by standard statistical operations, for example by estimating the probability distribution (data-based or model-based) or by calculating the time-dependent average or variance, for example using a time-sliding window. Morphological indicators: Indicators that emphasize different aspects of the morphology of the signal. Examples of this type of indicators are derivatives, curvature indicators or second degree derivative indicators, polynomial fit, overshoot estimation. The representation in the form of the well-known ADSR envelope can also be used as a morphological indicator. Indicators based on sudden changes: Indicators that evaluate changes in the signal that occur in a short time interval. For example, the moment the instrument comes into contact with a surface, the abrupt change in the signal can provide information about the hardness of the surface. Another example is the penetration of a needle into a layer, where the abrupt change resulting from the breakage of the tissue can be related to the elasticity of the tissue and its resistance property to the instrument tip. The abrupt change can be evaluated or calculated using a statistical hypothesis test such as the Hinkley and Page test or the CUSUM test.

Indicators in the frequency and time-frequency domain such as spectrum-based indicators corresponding to the stationary spectrum (e.g. a periodogram) or the time-frequency spectrum (e.g. using spectrogram, Wygner-Ville, CWT, time-varying autoregressive modeling, etc.). Indicators based on the instantaneous dominant frequency calculated from the time-frequency spectrum as the instantaneous dominant frequency, i.e. the frequency that contains the maximum energy in the time-frequency spectrum at any point in time. This can be calculated directly from the time-frequency matrix or parametrically via a time-variant model as in TVAR using pole estimation/tracking.

The concept of dominant frequency is only used in the literature for stationary spectral analysis (e.g. based on FFT) where time does not need to be considered. In music and speech signal processing, a similar concept is pitch. However, pitch refers to a “fundamental period” of the signal, and this denotes signals that are fairly stationary (the spectral properties do not change drastically with time). In the present case, due to the pronounced non-stationarity of the signals, an estimate of the frequency component that dominates over the others is made at each time point. By this extension of the concept to the instantaneous dominant frequency, dynamics and thus information can be obtained from transient or highly non-stationary signals.

The process step proposed for calculating the instantaneous dominant frequency, for example in the palpation example, is not known in the prior art.

Finally, envelope-based indicators are extracted indicators that correspond to the envelope of the signal. The envelope can be calculated, for example, using DWT or Hilbert transformation with or without homomorphic filtering.

Feature calculation: Four feature families can be extracted, which are divided into a total of ten sub-families. Each feature corresponds to a scalar number.

The feature calculation includes time-domain characteristics received as input of a time-domain indicator and used to calculate various families of characteristic scalar values. These include statistical features in the time domain for calculating classic statistical values from an indicator, for example average values, variance, kurtosis, skewness, etc. It also includes morphological features in the time domain for calculating morphological values from the indicators such as slopes, maximum slopes, curvatures, latencies, duration of segments and overshoots. The main objective of this family of features is to evaluate the morphology of the VA wave excitation over time resulting from an interaction event. This further includes abrupt change based features in the time domain, where this family of features aims to quantify the magnitude of change at the beginning or end of an interaction event or some abrupt changes that occur during an interaction event due to inhomogeneities in the tissue/surface. Higher values of these indicators could indicate harder tissue and vice versa.

Feature calculation includes spectral domain features to calculate feature scalar values from the stationary spectrum (vector) or from the time-frequency spectrum (matrix). This includes time-frequency spectrum event features, i.e. values associated with events that can be identified in the time-frequency spectrum. Examples of events are maximum spectral energies, abrupt changes in spectral energies, abrupt energy transition from one frequency to another, change from non-stationary to stationary frequency components, etc. This also includes spectral energy characteristics, i.e. characteristics that relate to the energies in the spectrum. They can be calculated as absolute or relative energies. The relative energies can be ratios between the energies in different bands (e.g. calculated from the spectral band decomposition) or relative to the total energy of the signal. This family of features provides information about the spectral distribution of the spectral energies involved in the interaction. For example, when a needle penetrates a tissue layer, high-frequency components are generally excited. Therefore, in this case, the energy ratios in the high frequency range are emphasized. During palpation, high frequencies are characteristic for interactions with hard tissue and low frequencies for interactions of the tool with soft tissue. This also includes statistical characteristics of the instantaneous dominant frequency for calculating classical statistical indicators from the instantaneous dominant frequency indicator, such as mean values and variance. Statistical values of instantaneous dominant frequency can be directly related to the homogeneity of the interacting surface or tissue. For example, a high variance of the instantaneous dominant frequency indicates that the interacting tissue is very inhomogeneous, and a low variance may be a characteristic of a smooth tissue or a tissue whose properties do not change significantly in space. This also includes dynamic features of the instantaneous dominant frequency, i.e. features that aim to describe the temporal behavior of the instantaneous dominant frequency. A dominant frequency can be tracked in the time domain or in its 2d complex representation. In the time domain, the dominant frequency acts like a signal whose dynamics (oscillatory, stationary, transient, etc.) can be extracted as a value. In the 2d complex representation, a dominant frequency can act like a particle moving in a 2d space. This particle contains a movement in time that can be quantified and converted into feature values.

Feature calculation also includes envelope-based features, i.e. features that are calculated directly from the envelope in the time domain or from a spectral transformation of the envelope. Sometimes these are two complex interactions that have a high degree of non-linear dynamics and are highly modulated (e.g. drilling or palpation of highly inhomogeneous structures), which can be simplified using the envelope of the signal. These include spectral envelope features, i.e. features that are calculated from the stationary or temporal frequency spectrum of the envelope. This also includes envelope-based features in the time domain, i.e. features that are calculated directly from the envelope indicator. They can provide important information about attenuation and irregularities in the interacting tissue. For example, the number of peaks and valleys in the envelope curve can provide information about unevenness caused by the interaction between the tool and the tissue/surface. These irregularities may be related to strong irregularities that may be inherent in the tissue or, in the case of palpation, related to underlying structures.

Feature calculation also includes deep learning-based features. Deep learning-based features are calculated by a multi-layer neural network that processes the data in a non-linear way. For this purpose, the spectral band decomposition data or that of the spectrum-based indicators can be fed as input data to the intended deep learning algorithm, e.g. multilayer perceptrons (MLPs), convolutional neural networks (CNNs), recurrent neural networks (RNNs), long short-term memory networks (LSTMs), autoencoders and restricted Boltzmann machines (RBMs). Each of these algorithms can automatically learn a set of higher level features based on the characterization of the input data.

The extracted features can be classified into a vector that is fed into a trained machine learning model that can be built using various architectures such as Support Vector Machine (SVM), k-Nearest Neighbors (kNN), Random Forest, neural networks and many other approaches. The trained machine learning model assigns the interaction events of the input signal VA to predefined classes. Examples of classes are: For palpation events, for example, these are different classes of softness and hardness or roughness and smoothness of the tissue. For insertion events, these are different classes of insertion tissues (e.g. fatty tissue, muscle tissue or connective tissue) or different classes of layers such as fascia, peritoneum, etc. For example, in laparoscopic procedures, the classes could be indicated for different tasks (grasping, cutting, palpation, suturing, etc.) or the classes could represent the different instruments that can be recognized when they come into contact with the tissue/surface (for example, grasper, forceps or scissors).

Finally, the identification of the different classes could be stored or displayed in a last step for different purposes such as monitoring, support, storage, to name a few.

### Specific Processing Examples

The generally described method can be optimized for specific applications by adapting individual process steps, for example for classifying tissue hardness/softness during palpation. The basic idea of the invention is to link signal characteristics with tissue/surface softness/hardness classes. For example, it is possible to divide tissue softness/hardness into ten classes, from class 1 for very soft tissue to class 10 for very hard tissue. A machine learning algorithm trained with tissues whose degree of softness/hardness is known can assign signal features to the defined classes, resulting in a machine learning trained model that is able to automatically categorize a palpation event into the different softness/hardness classes.

Specific processing examples are the subject of the following figures.

The device and method according to the invention have a number of advantages over the prior art. The detection and processing of vibroacoustic signals according to the invention make it possible to detect an intervention in much greater detail and with higher dynamics, since vibroacoustic signals are not subject to the limitations of visual methods, for example. The vibroacoustic waves also propagate outside the user's field of vision or line of sight. In addition, the device according to the invention does not require any active components in the sense of an ultrasonic probe, which therefore initially generate at least part of the signal to be captured themselves. The device according to the invention functions purely passively, i.e. it only picks up signals that are generated by the interaction. The recorded data also requires considerably less storage and can therefore be archived more easily.

The invention and the technical environment are explained in more detail below with reference to the figures. It should be noted that the figures show a particularly preferred embodiment of the invention. However, the invention is not limited to the embodiment shown. In particular, the invention includes any combination of the technical features listed in the claims or described in the description as being relevant to the invention, insofar as this is technically expedient.

It shows:

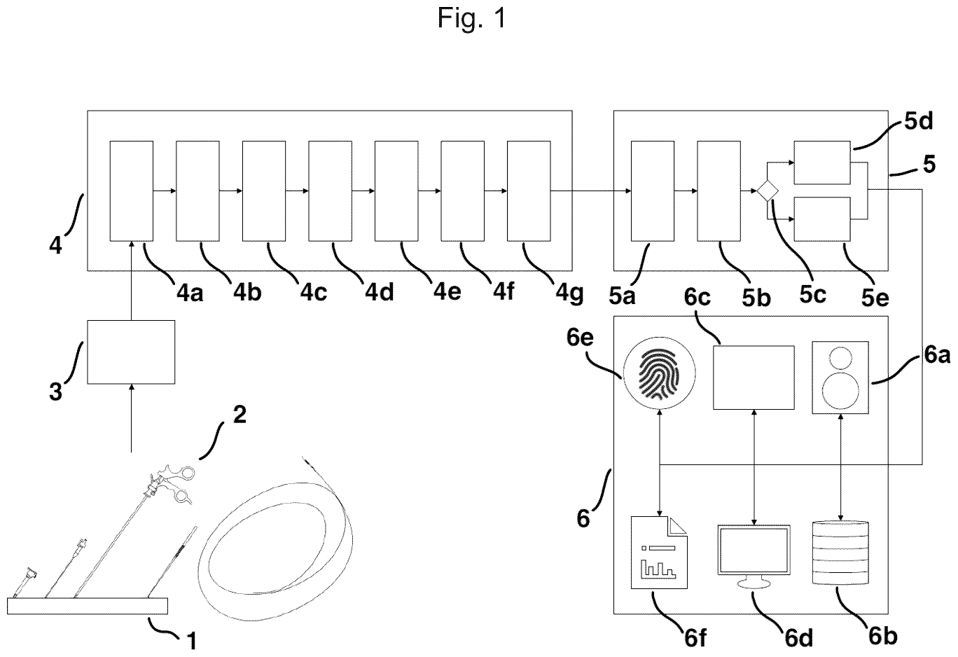

FIG. 1 a schematic view of the general structure of the device;

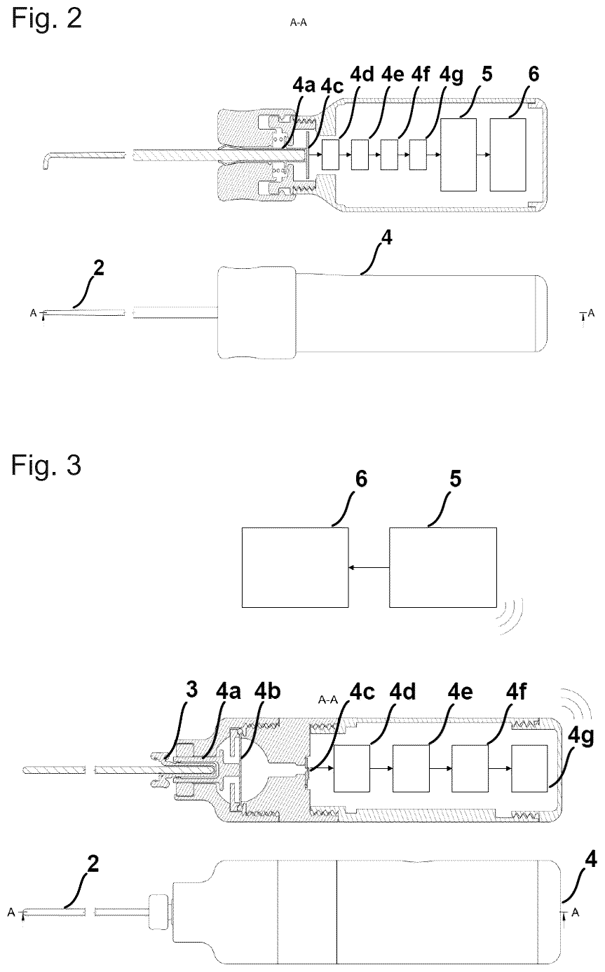

FIG. 2 the first embodiment of the device specifically for use with a hook probe;

FIG. 3 the second embodiment of the device specifically for use in palpation;

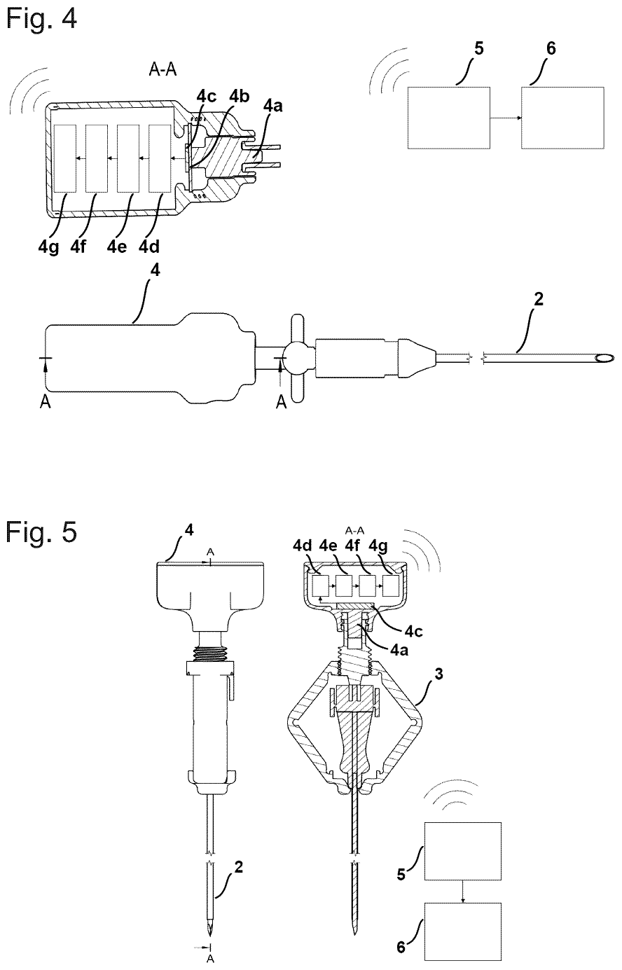

FIG. 4 the third embodiment of the device specifically for use with a Veres needle;

FIG. 5 the fourth embodiment of the device specifically for use with a biopsy needle;

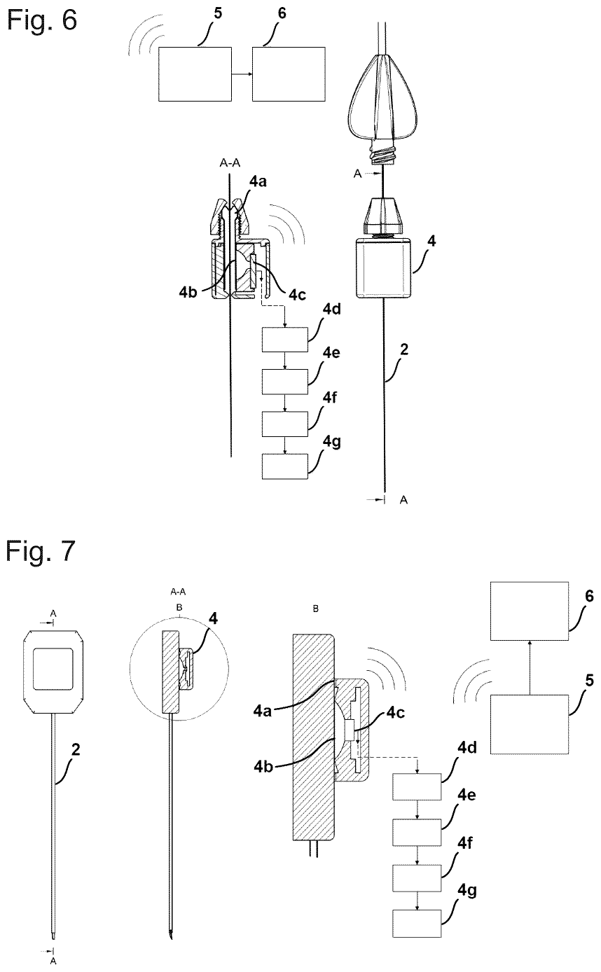

FIG. 6 the fifth embodiment of the device specifically for use with a guidewire of a catheter system;

FIG. 7 the sixth embodiment of the device specifically for use with a laparoscopic instrument in a robot-assisted intervention (RAS);

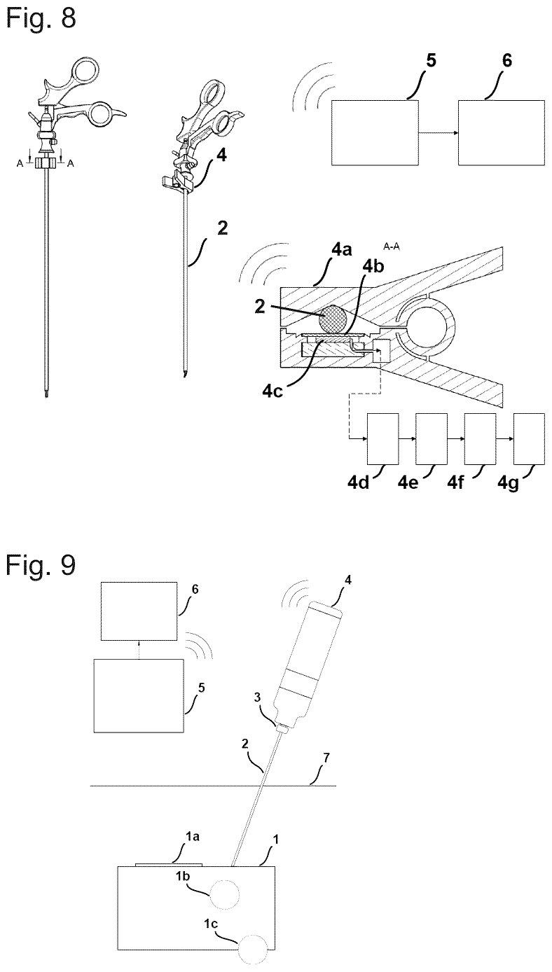

FIG. 8 the seventh embodiment of the device specifically for use with a rod-shaped laparoscopic instrument;

FIG. 9 Example of the use of the device according to the invention during palpation;

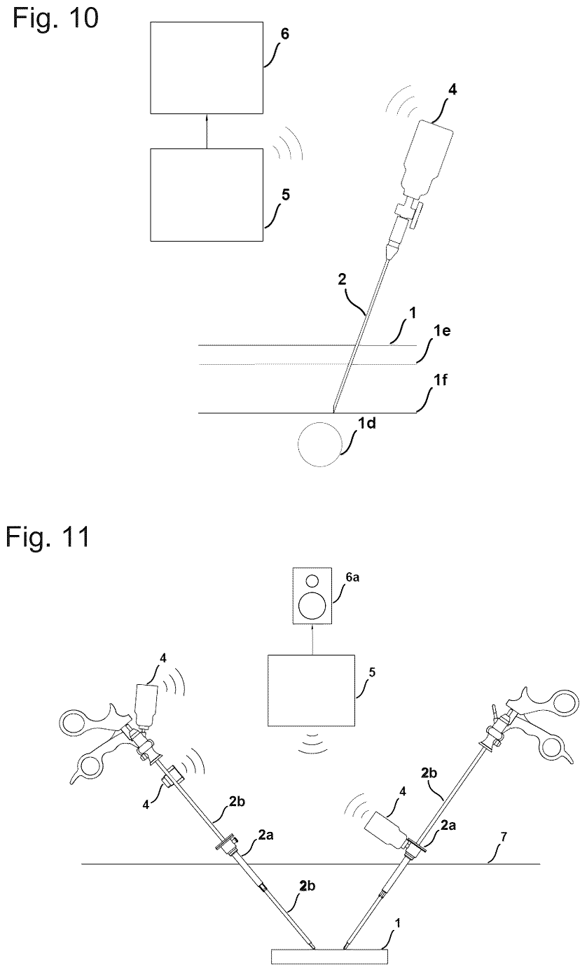

FIG. 10 Example of the use of the device according to the invention for the insertion of a puncture needle;

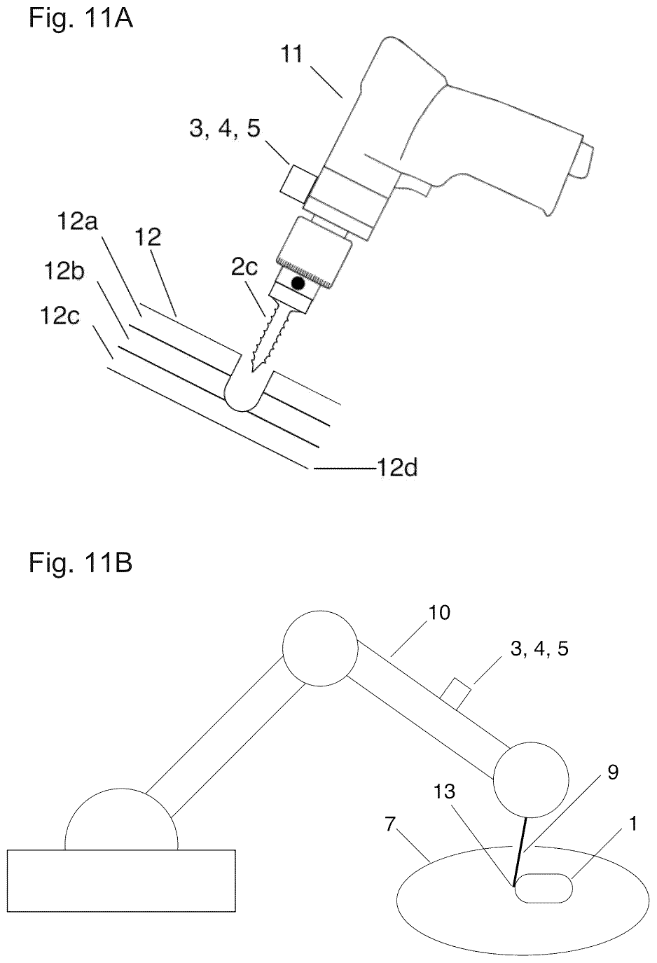

FIG. 11 Example of the use of several devices according to the invention during a laparoscopic intervention;

FIG. 11A Example of the use of the device according to the invention during surgical drilling;

FIG. 11B Example of the use of the device according to the invention for detecting collisions during robot-assisted interventions;

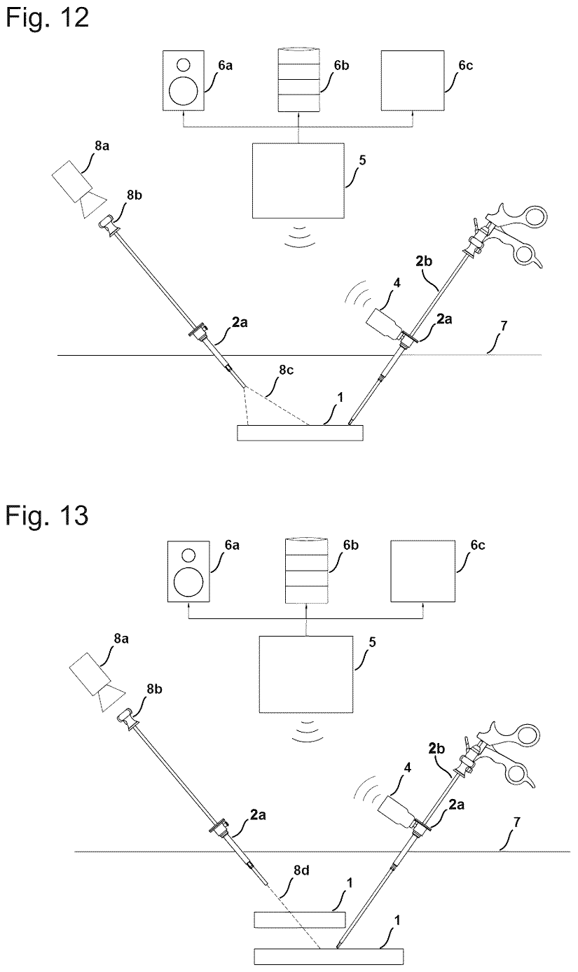

FIG. 12 Example of an application of the device according to the invention in a laparoscopic intervention for detecting interactions outside the field of view (FOV) of the endoscopic camera system;

FIG. 13 Example of an application of the device according to the invention during a laparoscopic intervention for detecting interactions outside the line of sight (LOS) of the endoscopic camera system;

FIG. 14 the flowchart of the general signal processing methodology or signal processing or data processing;

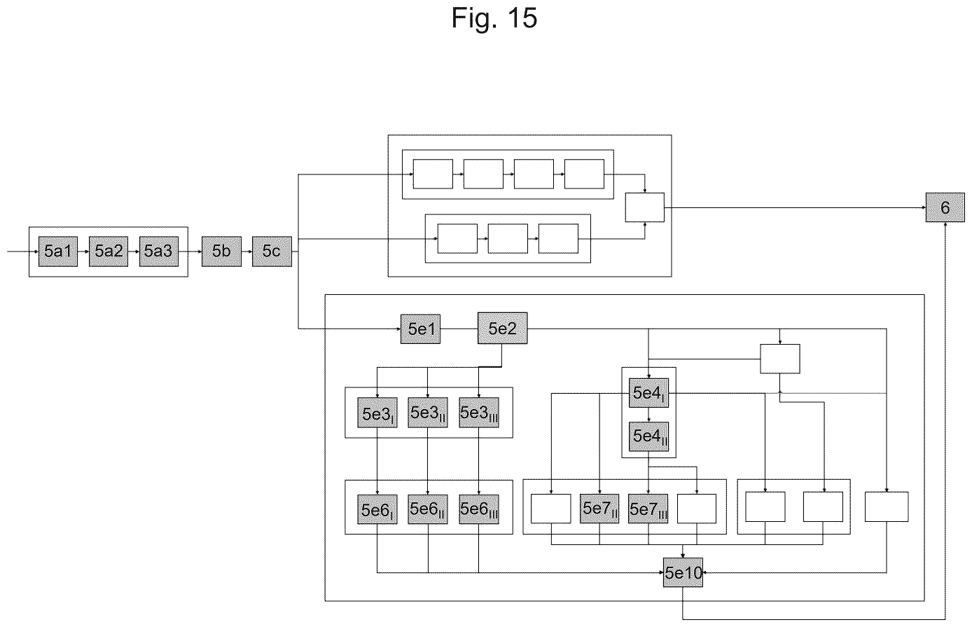

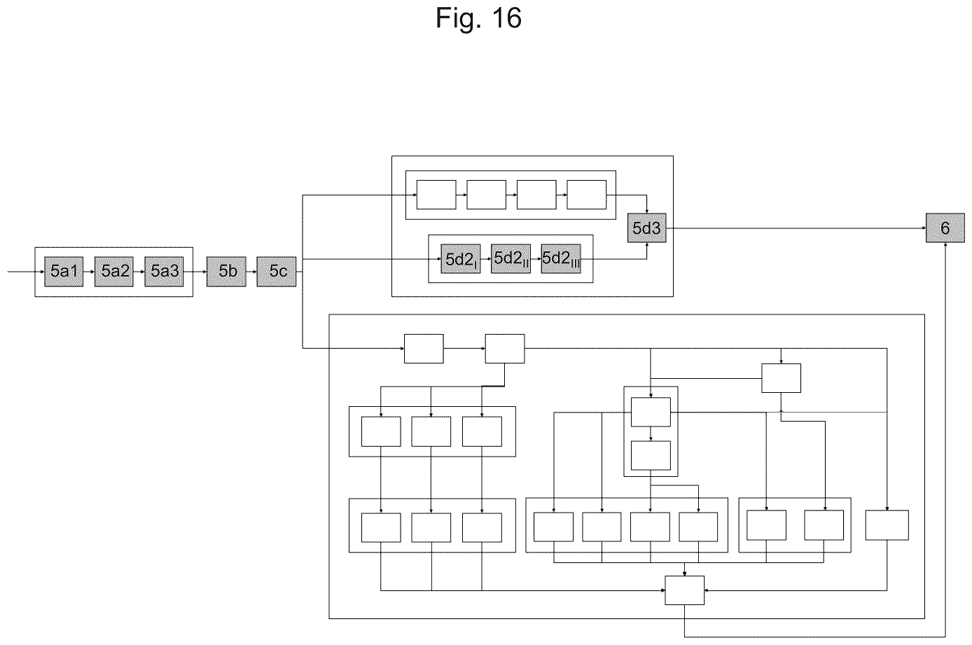

FIG. 15 the processing flowchart for a specific application for the classification of tissue hardness/tissue softness during palpation;

FIG. 16 the processing flow chart for a specific application for indirect testing of a needle puncture;

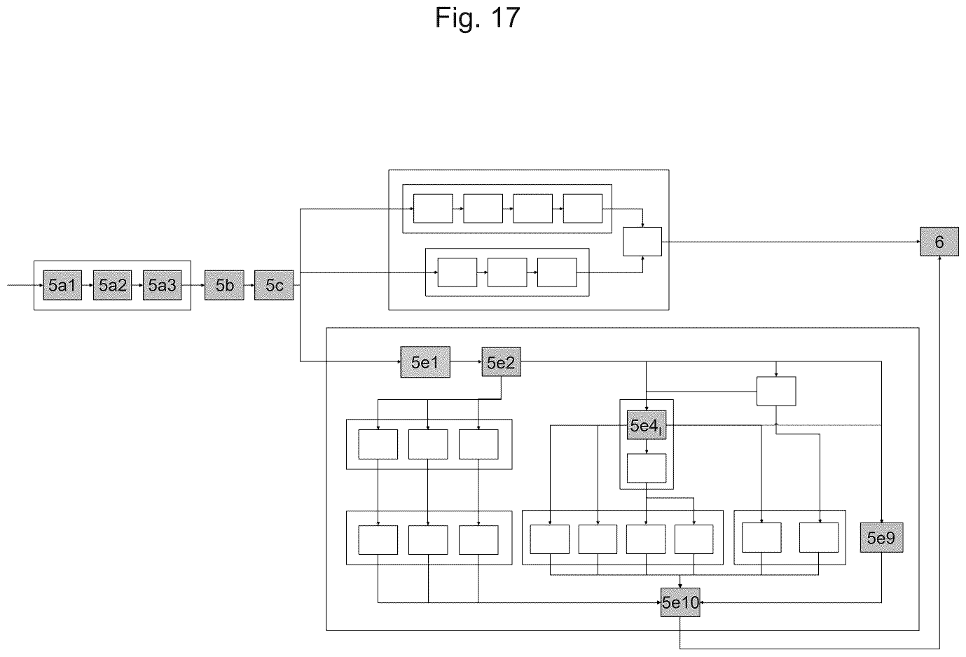

FIG. 17 the processing flow chart for a specific application for automatic detection of laparoscopic tools as they enter the surgical intervention through the trocar;

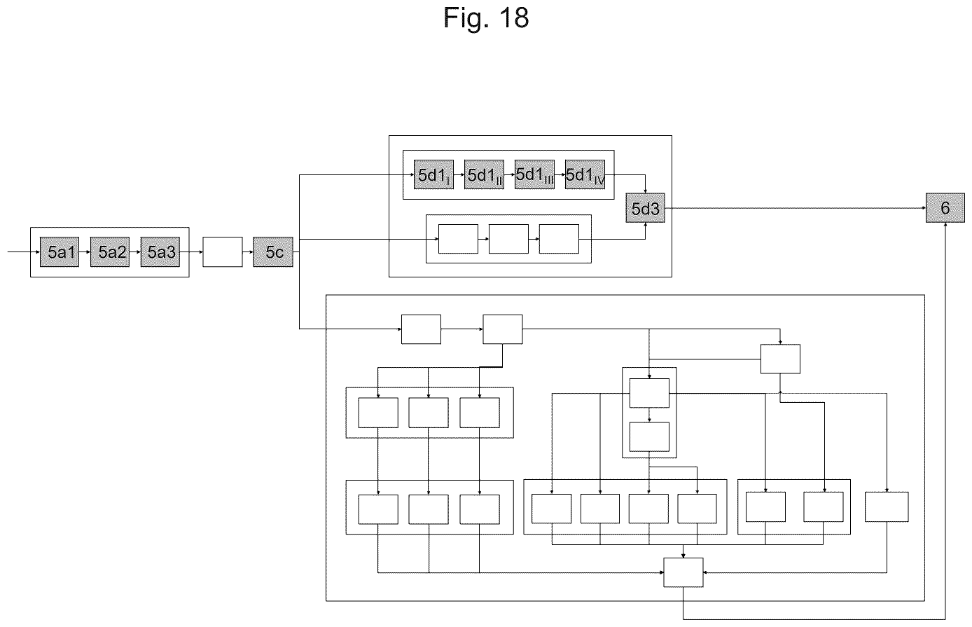

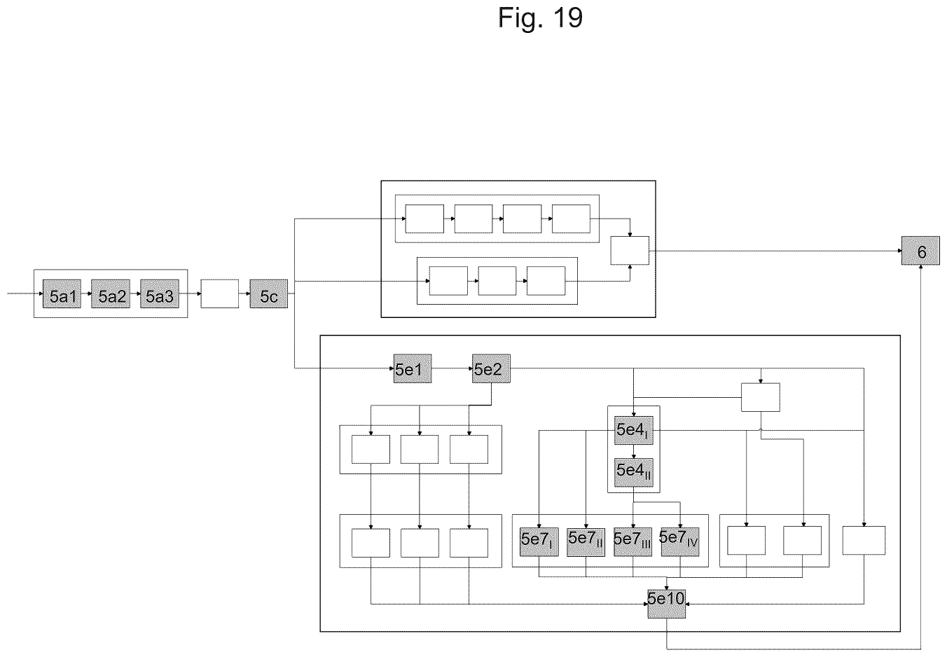

FIG. 18 the processing flow chart for a specific application for the evaluation of laparoscopic interventions, in which each interaction between tool and tissue occurring during the intervention is monitored and evaluated;

FIG. 19 the processing flow chart for a specific application for real-time vibroacoustic monitoring during electrosurgical interventions for tissue characterization;

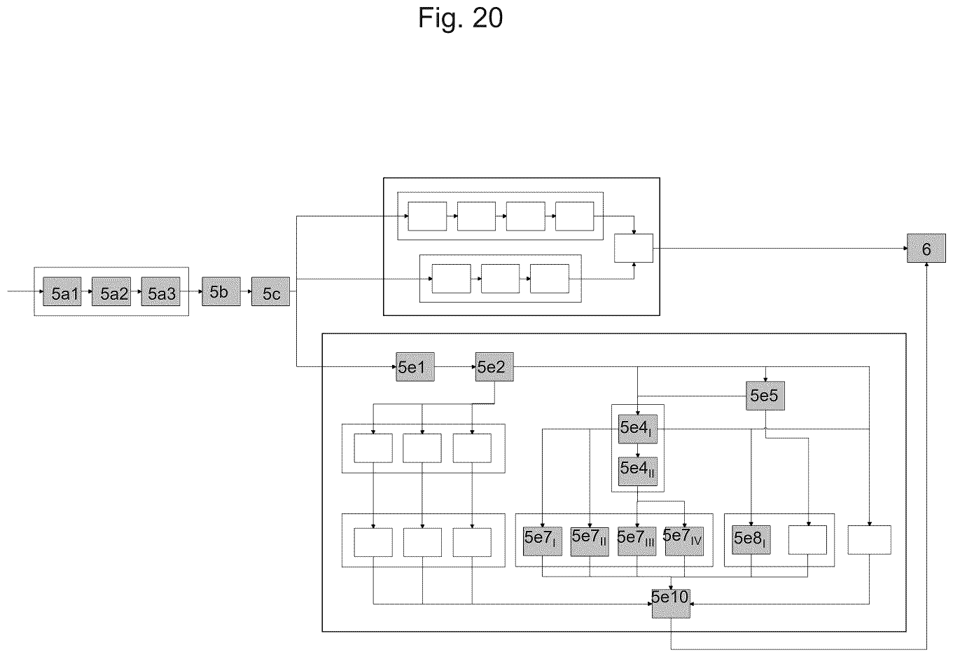

FIG. 20 the processing flow chart for a specific application for real-time vibroacoustic monitoring for precise bone drilling during surgical interventions;

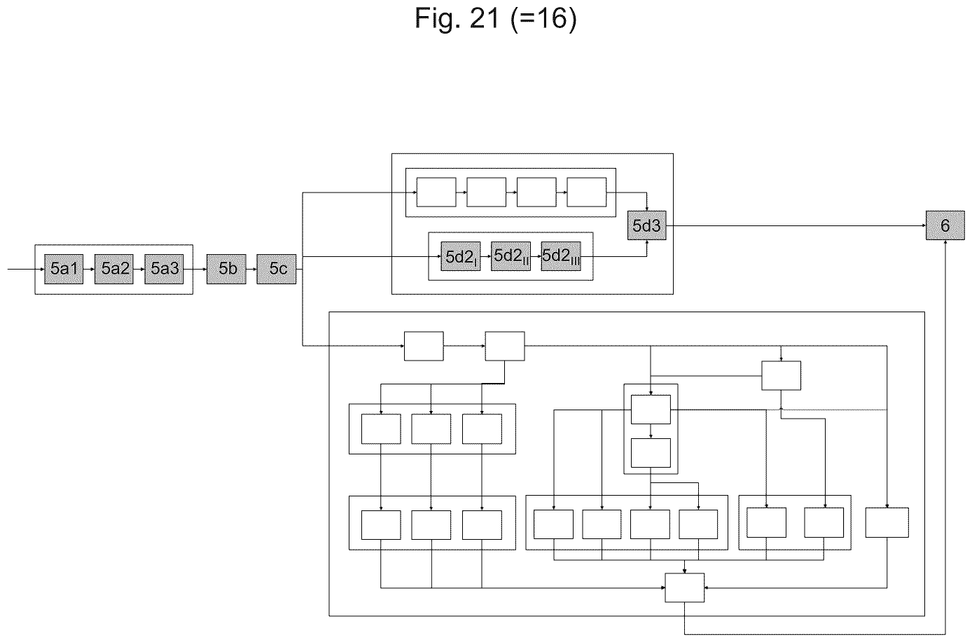

FIG. 21 the processing flow chart for a specific application for the detection and prevention of damage to pulsating structures in laparoscopic and robotic-assisted surgery;

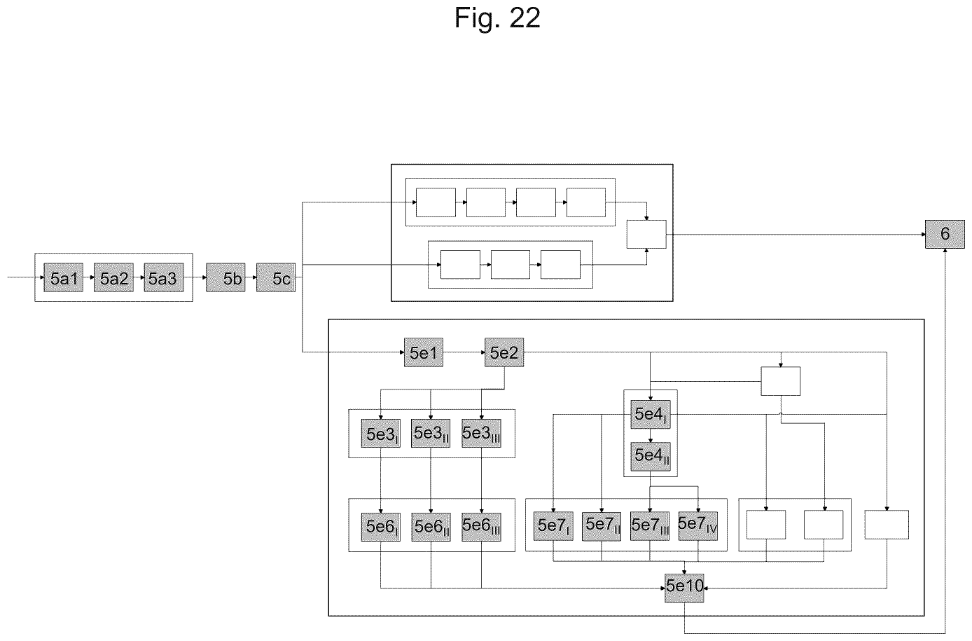

FIG. 22 the processing flow chart for a specific application for real-time detection and classification of collisions using the technology according to the invention.

FIG. 1 shows a schematic representation of the device according to the invention for capturing vibroacoustic signals and the corresponding method for evaluating vibroacoustic signals.

The device according to the invention comprises a signal acquisition device 4 with a coupling element 4a for fixing at least the signal acquisition device 4, for example at the proximal end of a medical instrument 2. A mechanical amplification mechanism 4b can be provided on the coupling element 4a, for example in the form of a membrane and/or a sound chamber. An acoustic or vibration sensor 4c, which is suitable for capturing vibroacoustic signals, is attached to the coupling element 4a or to the mechanical amplification mechanism 4b. The signals captured by the sensor 4c are optionally first processed on an analog level 4d and then fed to an analog-to-digital converter 4e. A controller 4f controls the processes and a communication unit 4g regulates the wireless or wired transmission of the digital data to the data processing/signal processing device 5.

The data processing/signal processing device 5 is intended to execute the program for the computer-implemented method according to the invention for processing the vibroacoustic signals or the corresponding digital data which the signal acquisition device 4 has transmitted to the data processing/signal processing device 5. The method comprises the steps of digital signal pre-processing 5a, identification of segments and regions of interest 5b and a subsequent audification 5d and/or 5c classification 5e. The specific sub-steps of the method are shown in FIG. 14 and may be the subject of all or part of the method. Specific process sequences are the subject of some of the following figures.

The results of the method with respect to the audification 5d and/or classification 5e process are transmitted wirelessly or by wire to the output device 6. The output device 6 either converts the result into an acoustic signal according to the requirements via a loudspeaker 6a or, if necessary, also stores the result in a database 6b, outputs the result to a virtual reality (VR) or augmented reality (AR) device 6c or visualizes the result on a screen 6d. It is also conceivable to output the result via a haptic device 6e, for example a vibration mechanism, or directly as an output in report form 6f.

FIG. 2 shows a first embodiment of the device according to the invention specifically for use with a hook probe. In this embodiment, the device comprises a signal acquisition device 4 with a coupling element 4a for fixing at least the signal acquisition device 4 to the proximal end of a medical instrument 2, namely a hook probe. In this embodiment, the proximal end of the coupling element 4a is directly connected to the at least one sensor and is suitable for transmitting vibroacoustic signals from the instrument 2 to the at least one sensor 4c with as little interference and loss as possible.

In the case of a direct connection, the at least one sensor 4c is connected directly to the proximal end of the coupling element 4a, wherein the sensor 4c then preferably is a piezo or crystal microphone.

The signals captured by the sensor 4c are optionally first processed at the analog level 4d and then fed to an analog-to-digital converter 4e. A controller 4f controls the processes and a communication unit 4g regulates the wireless or wired transmission of the digital data to the data processing/signal processing device 5.

The results are transmitted from the data processing/signal processing device 5 to the output device 6.

In this embodiment, the essential elements of the device according to the invention, namely the signal acquisition device 4, the data processing/signal processing device 5 and the output device 6 are preferably accommodated in a common housing.

FIG. 3 shows a second embodiment of the device according to the invention specifically for use in palpation. In this embodiment, the device according to the invention comprises a signal acquisition device 4 with a coupling element 4a for fixing at least the signal acquisition device 4 to the proximal end of a medical instrument 2, namely a palpation rod. The proximal end of the coupling element 4a is indirectly connected to the at least one sensor 4c and is suitable for transmitting vibroacoustic signals from the instrument 2 to the at least one sensor 4c with as little interference and loss as possible.

In this indirect connection, the proximal end of the coupling element 4a is not connected directly to a sensor 4c, but preferably to a membrane 4b, which transmits the vibroacoustic signals into a sound chamber adjoining the membrane 4b. The membrane 4b is suitable for amplifying the vibroacoustic signals transmitted by the coupling element 4a, whereby the sound chamber leads to an additional amplification of the signal. In principle, it is also conceivable that the vibroacoustic signals are transmitted directly, i.e. without an intermediate membrane 4b, from the coupling element 4a into the sound chamber.

In the case of an indirect connection, the at least one sensor 4c is preferably an air microphone.

The signals captured by the sensor 4c are optionally first processed on an analog level 4d and then fed to an analog-to-digital converter 4e. A controller 4f controls the processes and a communication unit 4g controls the wireless or wired transmission of the digital data to the data processing/signal processing device 5. The results are transmitted from the data processing/signal processing device 5 to the output device 6.

In this embodiment, the signal acquisition device 4 is preferably housed separately from the data processing/signal processing device 5 and the output device 6.

FIG. 4 shows a third embodiment of the device according to the invention specifically for use with a Veres needle. In this embodiment, the device according to the invention comprises a signal acquisition device 4 with a coupling element 4a for fixing at least the signal acquisition device 4 to the proximal end of a medical instrument 2, namely a Veres needle. The proximal end of the coupling element 4a is indirectly connected to the at least one sensor 4c and is suitable for transmitting vibroacoustic signals from the instrument 2 to the at least one sensor 4c with as little interference and loss as possible. In this indirect connection, the proximal end of the coupling element 4a is not connected directly to a sensor 4c, but preferably to a membrane 4b, which transmits the vibroacoustic signals amplified to a sensor 4c. The membrane 4b is suitable for amplifying the vibroacoustic signals transmitted by the coupling element 4a.

In this indirect connection, the at least one sensor 4c is preferably a piezo or crystal microphone.

The signals captured by the sensor 4c are optionally first processed at the analog level 4d and then fed to an analog-to-digital converter 4e. A controller 4f controls the processes and a communication unit 4g regulates the wireless or wired transmission of the digital data to the data processing/signal processing device 5. The results are transmitted from the data processing/signal processing device 5 to the output device 6.

In this embodiment, the signal acquisition device 4 is preferably housed separately from the data processing/signal processing device 5 and the output device 6.

FIG. 5 shows a fourth embodiment of the device according to the invention specifically for use with a biopsy needle. In this embodiment, the device comprises a signal acquisition device 4 with a coupling element 4a for fixing at least the signal acquisition device 4 to the proximal end of a medical instrument 2, namely a biopsy needle. In this embodiment, the proximal end of the coupling element 4a is directly connected to the at least one sensor and is suitable for transmitting vibroacoustic signals from the instrument 2 to the at least one sensor 4c with as little interference and loss as possible.

In this fourth embodiment, the biopsy needle 2 is arranged in a special adapter element 3 in order to be connected to the signal acquisition device in the best possible way via the coupling element 4a. The adapter element 3 comprises two angled elements which touch each other proximally and distally and which distally enclose the biopsy needle and proximally are provided for attachment to the coupling element 4a.

In the direct connection, the at least one sensor 4c is directly connected to the proximal end of the coupling element 4a, wherein the sensor 4c then preferably is a piezo or crystal microphone.

The signals captured by the sensor 4c are optionally first processed at the analog level 4d and then fed to an analog-to-digital converter 4e. A controller 4f controls the processes and a communication unit 4g regulates the wireless or wired transmission of the digital data to the data processing/signal processing device 5. The results are transmitted from the data processing/signal processing device 5 to the output device 6.

In this embodiment, the signal acquisition device 4 is preferably housed separately from the data processing/signal processing device 5 and the output device 6.

FIG. 6 shows a fifth embodiment of the device according to the invention specifically for use with the guidewire of a catheter system. In this embodiment, the device comprises a signal acquisition device 4 with a coupling element 4a for fixing at least the signal acquisition device 4 to the proximal end of a medical instrument 2, namely the guide wire of a catheter system. In this embodiment, the coupling element 4a passes through the signal acquisition device 4 from proximal to distal and thus forms a channel through which the guide wire of a catheter system is guided. A membrane 4b for amplifying the vibroacoustic signal is in contact with the channel-forming coupling element 4a, at least in sections.

Accordingly, the coupling element 4a is indirectly connected to the at least one sensor 4c and is suitable for transmitting vibroacoustic signals from the instrument 2 to the at least one sensor 4c with as little interference and loss as possible. In this indirect connection, the coupling element 4a is not connected directly to a sensor 4c, but to a membrane 4b, which transmits the vibroacoustic signals into a sound chamber adjoining the membrane 4b. The membrane 4b is suitable for amplifying the vibroacoustic signals transmitted by the coupling element 4a, whereby the sound chamber leads to an additional amplification of the signal. In principle, it is also conceivable that the vibroacoustic signals are transmitted directly, i.e. without an intermediate membrane 4b, from the coupling element 4a into the sound chamber.

In the case of an indirect connection, the at least one sensor 4c is preferably an air microphone.

The signals captured by the sensor 4c are optionally first processed on an analog level 4d and then fed to an analog-to-digital converter 4e. A controller 4f controls the processes and a communication unit 4g controls the wireless or wired transmission of the digital data to the data processing/signal processing device 5. The results are transmitted from the data processing/signal processing device 5 to the output device 6.

In this embodiment, the signal acquisition device 4 is preferably housed separately from the data processing/signal processing device 5 and the output device 6.

FIG. 7 shows a sixth embodiment of the device according to the invention specifically for use in robot-assisted surgery (RAS) and there in particular for attachment to the surface of a laparoscopic instrument 2. In this embodiment, the device according to the invention comprises a signal acquisition device 4 with a coupling element 4a for fixing at least the signal acquisition device 4 to the proximal end of a medical instrument 2, namely a laparoscopic instrument in RAS.

The proximal end of the coupling element 4a is indirectly connected to the at least one sensor 4c and is suitable for transmitting vibroacoustic signals from the instrument 2 to the at least one sensor 4c with as little interference and loss as possible. In this indirect connection, the proximal end of the coupling element 4a is not connected directly to a sensor 4c, but preferably to a membrane 4b, which transmits the vibroacoustic signals into a sound chamber adjoining the membrane 4b. The membrane 4b is suitable for amplifying the vibroacoustic signals transmitted by the coupling element 4a, whereby the sound chamber leads to an additional amplification of the signal. In principle, it is also conceivable that the vibroacoustic signals are transmitted directly, i.e. without an intermediate membrane 4b, from the coupling element 4a into the sound chamber.

In the case of an indirect connection, the at least one sensor 4c is preferably an air microphone.

The signals captured by the sensor 4c are optionally first processed on an analog level 4d and then fed to an analog-to-digital converter 4e. A controller 4f controls the processes and a communication unit 4g controls the wireless or wired transmission of the digital data to the data processing/signal processing device 5. The results are transmitted from the data processing/signal processing device 5 to the output device 6.

In this embodiment, the signal acquisition device 4 is preferably housed separately from the data processing/signal processing device 5 and the output device 6.

FIG. 8 shows a seventh embodiment of the device according to the invention specifically for use with a rod-shaped laparoscopic instrument. In this embodiment, the device according to the invention comprises a signal acquisition device 4 with a coupling element 4a for fixing at least the signal acquisition device 4 preferably to the proximal end of a medical instrument 2, namely a rod-shaped laparoscopic instrument. The coupling element 4a has a pincer-like design and can be fixed at one end to the rod-shaped section 2 of the laparoscopic instrument in the manner of a clothespin. In this clamp-like section of the coupling element 4a, a membrane 4b is provided at least in sections, which is in contact with the rod-shaped section 2 of the laparoscopic instrument. The membrane 4b is suitable for transmitting vibroacoustic signals from the instrument 2 to the at least one sensor 4c with as little interference and loss as possible. In this indirect connection, the proximal end of the coupling element 4a is not connected directly to a sensor 4c, but preferably to a membrane 4b, which transmits the vibroacoustic signals amplified to a sensor 4c. The membrane 4b is suitable for amplifying the vibroacoustic signals transmitted by the instrument 2 or, if applicable, also by the coupling element 4a.

In this indirect connection, the at least one sensor 4c is preferably a piezo or crystal microphone.

The signals captured by the sensor 4c are optionally first processed at the analog level 4d and then fed to an analog-to-digital converter 4e. A controller 4f controls the processes and a communication unit 4g regulates the wireless or wired transmission of the digital data to the data processing/signal processing device 5. The results are transmitted from the data processing/signal processing device 5 to the output device 6.

In this embodiment, the signal acquisition device 4 is preferably housed separately from the data processing/signal processing device 5 and the output device 6.

FIG. 9 shows the palpation in which the scanning of a tissue structure 1 is audified with the signal acquisition device 4 of the device according to the invention, which is connected to a scanning probe 2 via an adapter element 3, via the data processing device 5 and the output device 6.

FIG. 10 shows a percutaneous needle insertion in which the signal acquisition device 4 of the device according to the invention is connected to a Veres-type insufflation needle 2 and the penetration of individual tissue layers 1e, 1f, 1d is audified via the data processing device 5 and the output device 6.

FIG. 11 shows a laparoscopic intervention with several signal acquisition devices 4, each connected to the instruments 2 used to monitor the tissue-instrument interactions 1 during the intervention within the body 7.

FIG. 11A shows a device according to the invention for a specific application for real-time vibroacoustic monitoring for precise bone drilling and screwing during surgical procedures.

The signal acquisition device 4 is connected to the data processing/signal processing device 5 via an optional adapter element 3 on the medical instrument, here on a part of a surgical drill machine 11, for example also the drill chuck. The surgical drill machine 11 comprises a drill 2c, which can be used to drill into bone material 12. The different bone layers with different bone densities 12a to 12d can be detected by the vibroacoustic signals they emit implicitly by the drill and the breakthrough of the final bone layer.

Further details can be found in the description of FIG. 19.

FIG. 11B shows the data processing flowchart for a specific application for real-time vibroacoustic monitoring to detect collisions during surgical procedures.

The signal acquisition device 4 is connected to the data processing/signal processing device 5 via an optional adapter element 3 on the medical instrument, in this case a surgical robot or robotic arm 10. The surgical instrument 9, which is attached to the robotic arm 10, should preferably not collide with the body 7 or the tissue 1. Even the smallest collision 13 can be captured using vibroacoustic signals and reported to the user or the robot (arm) as a control signal.

Further details can be found in the description of FIG. 22.

FIG. 12 shows a laparoscopic intervention in which the signal acquisition device 4 is connected to the laparoscopic port (trocar) 2a and indirectly captures the interaction of the inserted laparoscopic instrument 2b in order to recognize and use interactions outside the field of view 8c (FOV) of the endoscopic camera system 8a.

FIG. 13 shows a laparoscopic intervention in which the signal acquisition device 4 is connected to the laparoscopic port (trocar) 2a and indirectly detects the interaction of the inserted laparoscopic instrument 2b in order to recognize and use interactions outside the line of sight 8d (LOS) of the endoscopic camera system 8a.

FIG. 14 shows the basic flowchart of the method according to the invention for data processing of vibroacoustic signals. Not all process steps are used or necessary for all applications. The following figures contain special applications of the method. Accordingly, the individual possible steps are briefly explained below. The exact processing flow is shown in the figure.

The computer-implemented method according to the invention basically comprises, in a first step not shown, the provision of existing digitized vibroacoustic signals.

Accordingly, the basic process sequence of the computer-implemented method according to the invention comprises the following steps:
(0) Providing an existing digitized vibroacoustic signal;
(I) Signal pre-processing of the vibroacoustic signal (5a); and
(II) Identification of the segments/regions of interest (5b); and
(III) Audification (5d); and/or (5c)
(IV) Classification of the segments/regions of interest (5e).

Optionally, for example the output, further processing, storage, etc. (6) of the classified segments/regions takes place in a further step (V).

The pre-processing (Vorverarbeitung) (5a) aims to attenuate the various interfering noises in the VA signal and to amplify the interaction events between the instrument tip and shaft and tissue. First, the continuous components (DC) are removed from the VA signal 5a1. Then a background noise reduction step 5a2 is performed. This can be done, for example, by spectral gating methods. Finally, a signal enhancement step 5a3 is performed to improve the dynamics and the frequency components associated with the interactions. This can be done with linear filters, adaptive filters or wavelet-based filters. In wavelet-based filters, the discrete wavelet transform (DWT) can be used to decompose the signal into different scales and then reconstruct the signal based on the scales where events of interest occur, these can be high energy interactions for example.

The identification of segments/regions of interest (Identifizierung von Segmenten/Regionen von Interesse) 5b can be used to segment segments and regions of interest in the signal where important interaction events between instrument and tissue/surface occur. This can be used to perform an audification or classification focusing only on the relevant interaction information. For this purpose, the onset and shift of a possible interaction event can be determined using, for example, abrupt change detection algorithms (for example, the Hinkley Page hypothesis test) or derivative detection algorithms.

### Non-Exclusive OR Operator (Nicht-Ausschlieβlicher ODER-Operator) 5c

This operator indicates the possibility of following the path of audification and/or the path of classification. For some applications only audification can be provided as feedback and for some other applications only classification is necessary. There are also applications that require an integration of classification and audification of interaction events.

### Audification (Audifikation) 5d

Direct audification (direkte Audifikation) 5d1: This is a direct conversion of the VA signal into an audible sound domain using various standard processing tools such as filtering, gain operators or effect generation. It can include four main sub-steps:
   Filtering (Filterung des Signals) 5d1_{I}: The main goal of this step is to enhance the frequency components of signal events or signal segments of interest related to tip/shaft and tissue/surface interactions significant to a particular procedure. This filtering stage can be performed using different techniques, such as linear filtering, adaptive filtering or filtering generated using any type of signal decomposition techniques, such as continuous wavelet transform (CWT), discrete wavelet transform (DWT), empirical mode decomposition (EMD) or autoregressive modeling. The signal is decomposed into different scales, modes or dynamics (depending on the technique used) and then reconstructed using scales/modes/dynamics where high energy interaction events are present.
   Volume adjustment (Lautstärkeanpassung) 5d1_{II}: This consists of applying a gain to the signal to increase the volume. It can also be used to limit excessively loud events or saturated audio signal segments.
   Dynamic processing (Dynamikbearbeitung) 5d1_{III}: With this step the dynamic characteristics of the audio signal are modified. It allows to set a ratio between the input levels and the output levels. This can be done in a single frequency band or in several frequency bands for independent, parallel dynamic processing.
   Effects generation (Erzeugung von Effekten) 5d1_{IV}: The aim is to manipulate the sound of the audio signal. Common effects that can be used here are reverberation, reverb, convolution, echo, chorus, time stretching, transposition, harmonizer.
Indirect audification (indirekte Audifikation) 5d2: This step involves linking features of the VA signal with auditory parameters for the purpose of data visualization. It consists of extracting features that are then converted into an auditory representation by mapping the features to different parameters of the displayed sound. It comprises three main sub-steps:
   Feature extraction (Merkmalsextraktion) 5d2_{I}: In the first stage, different features or attributes are extracted from the signal. Different types of features can be extracted from the signal segment, such as features in the time domain, frequency domain or time-frequency domain. Examples of features in the time domain are signal strengths or signal energies, statistical features such as signal mean or signal variance and morphological features. For frequency domain features, estimates of the signal spectrum can be performed using various spectral estimation methods such as FFT, AR modeling, etc. For time/frequency domain features, methods such as short-time Fourier transform (STFT), Mel frequency cepstrum, Wigner-Ville distribution, continuous wavelet transform (CWT), discrete wavelet transform (DWT), empirical mode decomposition (EMD), time-varying autoregressive modeling, cyclostationarity features, etc. can be used.
   Mapping (Mapping) 5d2_{II}: The features are then converted into audio parameters that are used to generate an auditory representation. This can be done
      by direct mapping of the extracted features via parameter mapping sonification, in which, for example, changes in signal strength can be mapped to changes in the pitch of the resulting acoustic representation;
      using a dynamic model that describes the temporal evolution of a system excited by the features, where the model is intended to simulate a physical sound source
      using decision rules, clustering methods or a trained machine learning model that uses the features as inputs and generates classified events as outputs, which are then used to parameterize the sound.
   Sound synthesis (Klangsynthese) 5d2_{III}: Generation of sound features.
Sound projection (Tonprojektion) 5d3: Generates everything necessary for the transmission and/or output of the generated sound output at the output device (6), via one or more loudspeakers or headphones.

### Classification of the Segments/regions of Interest (klassifizierung) 5e

Transient and stationary dynamics identification (Identifizierung transienter und stationärer Dynamik) 5e1: The VA signals resulting from the interactions between the instrument and the surface exhibit very complex dynamics. A single interaction event can exhibit a mixture of transient and stationary dynamics in a very short time interval. For example, in a probing event, the moment the tip of the instrument comes into contact with the VA excitation, this results in an extremely short transient response, followed by a steady-state response resulting from the effective probing process. Inhomogeneities of the surface can lead to collisions of the tip with the surface, which in turn leads to a mixture of transient and steady-state dynamics. At the moment the palpation stops, the contact stop can also generate transient dynamics, so that a series of transient-stationary dynamics can occur in a short time interval. This complexity means that the signal cannot be processed using standard or classical signal processing algorithms, which are normally used for speech or musical tones, whose acoustic responses can be quite stable in a short to medium time interval.
Spectral band decomposition (Spektralbandzerlegung) 5e2: In this step, the VA signal is decomposed into several narrowband signals by splitting the overall frequency band of the signal into several sub-frequency bands. The main goal is to later process different aspects of the signal separately. We assume that an image texture is composed of several dynamics that represent irregularity properties of the texture such as smoothness or roughness. Therefore, the signals can be decomposed into several dynamics that can represent, for example, the levels of irregularities in the interaction. The number of frequency bands into which the signal is decomposed depends on whether the signal is generated by insertion, palpation or some other type of interaction between the instrument and the tissue/surface. The number of bands can vary between 2 and 10 frequency sub-bands. The decomposition can be performed using different methods, e.g. filter banks, continuous wavelet transform (CWT), discrete wavelet transform (DWT), pole tracking and time-variant autoregressive modeling, empirical mode decomposition (EMD), etc.
Indicators computation (Berechnung von Indikatoren): Before the feature extraction step, it is necessary to first extract indicators from the signals. This indicator computation normally receives one or more signals as input and provides one or more signals or matrices as output. Three types of indicators are proposed part of the signal processing method according to the invention:
   Time domain indicators (Zeitbereichsindikatoren) 5e3: These are indicators that are calculated directly from the time series. Three types of time domain indicators can be calculated:
      Statistical indicators (statistische Indikatoren) 5e3_{I}: Indicators calculated by standard statistical operations, e.g. by estimating the probability distribution (data-based or model-based) or by calculating the time-dependent average or the variance, e.g. using a time-sliding window.
      Morphological indicators (morphologische Indikatoren) 5e3_{II}: Indicators that emphasize different aspects of the morphology of the signal. Examples of this type of indicators are derivatives, curvature indicators or second degree derivative indicators, polynomial fit, overshoot estimation. The representation of the ADSR envelope can also be used as a morphological indicator.
      Abrupt change-based indicators (auf plötzlichen Änderungen basierende Indikatoren) 5e3_{III}: Indicators that evaluate changes in the signal that occur in a short time interval. For example, the moment the instrument comes into contact with a surface, the abrupt change in the signal can provide information about the hardness of the surface. Another example is the penetration of a needle into a layer, where the abrupt change resulting from the breakage of the tissue can be related to the elasticity of the tissue and its resistance property to the instrument tip. The abrupt change can be evaluated or calculated for example using a statistical hypothesis test such as the Hinkley and Page test or the CUSUM test.
   Frequency and time-frequency domain indicators (Indikatoren im Frequenz- und Zeit-Frequenz-Bereich) 5e4:
      Spectrum-based indicators (spektrumbasierte Indikatoren) 5e4_{I}: Indicators corresponding to the stationary spectrum (e.g. a periodogram) or the time-frequency spectrum (e.g. using spectrogram, Wygner-Ville, CWT, time-variant autoregressive modeling, etc.).
      Instantaneous dominant frequency-based indicators (Indikatoren auf der Grundlage der momentanen dominanten Frequenz) 5e4_{II}: Indicators calculated from the time-frequency spectrum as the instantaneous dominant frequency, i.e. the frequency that contains the maximum energy in the time-frequency spectrum at any point in time. This can be calculated directly from the time-frequency matrix or parametrically via a time-variant model as in TVAR using pole estimation/tracking.
   Envelope-based indicators (hüllkurvenbasierte Indikatoren) 5e5: Extracted indicators that correspond to the envelope of the signal. The envelope can, for example, be calculated using DWT or Hilbert transform with or without homomorphic filtering.
Features computation (Merkmalsberechnung): Four feature families can be extracted, which can be subdivided into a total of ten sub-families. Each feature corresponds to a scalar number.
   Time-domain features (Zeitbereichsmerkmale) 5e6: You receive a time-domain indicator as input and calculate various families of feature scalar values.
      Time-domain statistical features (statistische Merkmale im Zeitbereich) 5e6_{I}: Calculation of classic statistical values from an indicator, e.g. average values, variance, kurtosis, skewness, etc.
      Time-domain morphological features (morphologische Merkmale im Zeitbereich) 5e6_{II}: Calculation of morphological values from the indicators such as slopes, maximum slopes, curvatures, latencies, duration of segments and overshoots. The main objective of this family of features is to evaluate the morphology of the VA wave excitation over time resulting from an interaction event.
      Time-domain abrupt change based features (auf abrupten Verdänderungen basierende Merkmale im Zeitbereich) 5e6_{III}: This family of features aims to quantify the magnitude of change at the beginning or at the end of an interaction event or some abrupt changes that occur during an interaction event due to inhomogeneities in the tissue/surface. Higher values of these indicators could indicate harder tissue and vice versa.
   Spectral domain features (Spektralbereich-Merkmale) 5e7: Calculation of feature scalar values from the stationary spectrum (vector) or from the time-frequency spectrum (matrix).
      Time frequency spectrum events features (Zeit-Frequenz-Spektrum-Ereignis-Merkmale) 5e7_{I}: Values associated with events that can be identified in the time-frequency spectrum. Examples of events are maximum spectral energies, abrupt changes in spectral energies, abrupt energy transition from one frequency to another, change from non-stationary to stationary frequency components, etc.
      Spectral energy features (Spektralenergiemerkmale) 5e7_{II}: Features that relate to the energies in the spectrum. They can be calculated as absolute or relative energies. The relative energies can be ratios between the energies in different bands (e.g. calculated from 5e2) or relative to the total energy of the signal. This family of features provides information about the spectral distribution of the spectral energies involved in the interaction. For example, when a needle penetrates a tissue layer, high-frequency components are generally excited. Therefore, in this case, the energy ratios in the high frequency range are emphasized. In palpation, for example, high frequencies are characteristic of interactions with hard tissue and low frequencies for interactions of the tool with soft tissue.
      Instantaneous dominant frequency statistical features (statistische Merkmale der momentanen dominanten Frequenz) 5e7_{III}: Calculation of classical statistical indicators from the instantaneous dominant frequency indicator, such as mean values and variance. Instantaneous dominant frequency statistical values can be directly related to the homogeneity of the interacting surface or tissue. For example, a high variance of the instantaneous dominant frequency indicates that the interacting tissue is very inhomogeneous, and a low variance may be a characteristic of a smooth tissue or a tissue whose properties do not change significantly in space.
      Instantaneous dominant frequency dynamical features (dynamische Merkmale der momentanen dominanten Frequenz) 5e7_{IV}: Features that aim to describe the temporal behavior of the instantaneous dominant frequency. A dominant frequency can be tracked in the time domain or in its time course of the complex number representation 2d. In the time domain, the dominant frequency acts like a signal whose dynamics (oscillating, stationary, transient, etc.) can be extracted as values. In the 2d complex representation, a dominant frequency can act as a particle moving in a 2d space. This particle contains a movement in time that can be quantified and converted into feature values.
   Envelope-based features (hüllkurvenbasierte Merkmale) 5e8: Features that are calculated directly from the envelope in the time domain or from a spectral transformation of the envelope. Sometimes these are two complex interactions that have a high degree of non-linear dynamics and are highly modulated (for example, drilling or palpation of highly inhomogeneous structures), and they can be simplified using the envelope of the signal.
      Spectral envelope features (spektrale Hüllkurvenmerkmale) 5e8_{I}: Features calculated from the stationary or temporal frequency spectrum of the envelope. These features are used to extract non-linear features of the interaction.
      Time-domain envelope-based features (hüllkurvenbasierte Merkmale im Zeitbereich) 5e8_{II}: Features calculated directly from the envelope indicator. They can provide important information about attenuation and irregularities in the interacting tissue. For example, the number of peaks and valleys in the envelope can provide information about irregularities caused by the interaction between the tool and the tissue/surface. These irregularities may be related to strong irregularities that may be inherent to the tissue or, in the case of palpation, may be related to underlying structures.
   Deep learning based features (Deep-Learning-basierte Merkmale) 5e9: Deep learning based features are calculated by a multilayer neural network that processes the data in a non-linear way. For this purpose, the spectral band decomposition (5e2) or the spectrum-based indicators (5e4_{I}) can be fed as input data to the intended deep learning algorithm, e.g. multilayer perceptrons (MLPs), convolutional neural networks (CNNs), recurrent neural networks (RNNs), long short-term memory networks (LSTMs), autoencoders and restricted Boltzmann machines (RBMs). Each of these algorithms can automatically learn a set of higher level features based on the characterization of the input data.
Machine learning trained model (trainiertes Modell de maschinellen Lernens) 5e10: The extracted features can be classified into a vector that is fed into a trained machine learning model that can be created using various architectures such as Support Vector Machine (SVM), k-Nearest Neighbors (kNN), Random Forest, neural networks and many other approaches. The trained machine learning model assigns the interaction events of the input signal VA to predefined classes. Examples of classes are
   For palpation events: different classes of softness/hardness or roughness/smoothness of the tissue.
   For insertion events: different classes of insertion tissue (fat, muscle, connective tissue, etc.) or different classes of layers such as fascia, peritoneum, etc.
   For example, for laparoscopic procedures, the classes could be indicated for different tasks (grasping, cutting, palpation, suturing, etc.), or the classes could be the different instruments that can be recognized when they come into contact with the tissue/surface (grasper, forceps, scissors, etc.)
Finally, the identification of the different classes could also be stored or displayed in the output device 6 for different purposes such as monitoring, support, storage, etc.

FIG. 15 shows in gray the blocks used for the method according to the invention in connection with a palpation from the general processing chart of FIG. 14.

The generally described method can be optimized for specific applications by adapting individual method steps, for example for classification of events such as tissue hardness/softness during palpation. The basic idea of the invention is to link signal characteristics with tissue or surface softness or hardness classes. For example, it is possible to divide tissue softness/hardness into different classes, for example from class 1 for very soft tissue to class 10 for very hard tissue. A machine learning algorithm that has been trained with tissues whose degree of softness/hardness is known can assign signal features to the defined classes, resulting in a machine learning trained model that is able to automatically classify a palpation event into the different softness/hardness classes.

The method according to the invention for processing vibroacoustic signals generated during palpation is used in particular for feature extraction and classification. In particular, the method provides a translation algorithm with the aid of which the vibroacoustic signals can be translated into information relevant to the task. Such information can be, for example, the representation of properties of the target structure or guidance information for medical instruments.

A preferred method for signal or data processing, for example for classifying tissue hardness during palpation, comprises the following steps:
(0) Providing a digitized vibroacoustic signal;
(I) Signal pre-processing of the known vibroacoustic signal (5a);
(II) Identification of segments/regions of interest (5b); and
(III) Classification (5e) of palpation events into one of the specified classes, in particular into a softness/hardness class.

Optionally, in a step (IV), the classification is output by an output device 6 in haptic and/or acoustic and/or optical form.

The numbering of the process steps refers to FIG. 14.

The idea of the invention thus consists in particular of capturing vibroacoustic signals and breaking them down into various features. These features can then be linked to specific tissue/surface softness/hardness classes.

Accordingly, the method for feature extraction and classification during palpation comprises the following steps:
(0) Providing a digitized vibroacoustic signal;
(I) Signal pre-processing of the known vibroacoustic signal (5a), comprising the process steps
   i. Removal of continuous components (DC) from the VA signal (5a1);
   ii. Reduction of background noise (5a2);
   iii. Signal enhancement (5a3);
(II) Identification of segments/regions of interest (5b); and
(III) Classification of the vibroacoustic signals generated during palpation into one of the specified classes, in particular a softness/hardness class (5e), comprising the process steps of
   i. Identification of transient and stationary dynamics (5e1);
   ii. Spectral band decomposition (5e2);
   iii. Calculation of time domain indicators (5e3), namely:
      1. Statistical indicators (5e3_{I});
      2. Morphological indicators (5e3_{II});
      3. Indicators based on sudden changes (5e3_{III});
   iv. Time domain characteristics (5e6), namely:
      1. Statistical indicators (5e6_{I});
      2. Morphological indicators (5e6_{II});
         3. Indicators based on sudden changes (5e6_{III});
   v. Indicators in the frequency and time-frequency domain (5e4), namely:
      1. Identification of spectrum-based indicators (5e4_{I});
      2. Indicators based on instantaneous dominant frequency (5e4_{II}); and
   vi. Spectral domain features (5e7), namely:
      1. Time-frequency spectrum event features (5e7_{II});
      2. Statistical features of instantaneous dominant frequency (5e7_{III});
   vii. Classification by a machine learning model (5e10).

Optionally, in a step (IV), the classification is output by an output device 6 in haptic and/or acoustic and/or optical form.

The numbering of the process steps refers to FIG. 14.

The concept of dominant frequency is only used in the literature for stationary spectral analyses (e.g. based on the FFT), where time does not have to be taken into account. In music and speech signal processing, a similar concept is pitch. However, pitch refers to a “fundamental period” of the signal, and this denotes signals that are fairly stationary (the spectral properties do not change drastically with time). In the present case, due to the pronounced non-stationarity of the signals, an estimate of the frequency component that dominates over the others is made at each time point. By this extension of the concept to the instantaneous dominant frequency 5e7_{III}, dynamics and thus information can be obtained from transient or highly non-stationary signals.

The process step proposed for calculating the instantaneous dominant frequency, for example during palpation, is not known in the prior art.

FIG. 16 shows the data processing flowchart for a specific application for indirect auditing of a needle puncture, for example with a biopsy needle, a Veres needle or other needle.

A preferred computer-implemented method for indirect audification for indirect auditing of a needle puncture comprises the following steps:
(0) Providing a digitized vibroacoustic signal;
(I) Signal pre-processing the known vibroacoustic signal (5a), comprising the process steps of
   i. Removing continuous components (DC) from the VA signal (5a1);
   ii. Background noise reduction (5a2);
   iii. Signal enhancement (5a3);
(II) Identification of segments/regions of interest (5b); and
(III) Indirect audification (5d2), comprising the process steps of
   i. Feature extraction (5d2_{I});
   ii. Mapping of the extracted features (5d2_{II});
   iii. Generation of sound characteristics (5d2_{III}); and
(IV) Transfer (5d3) to the output device (6).

The numbering of the process steps refers to FIG. 14.

This is a specific example of a processing algorithm for generating an audible alarm signal at each major event during the insertion of, for example, a Veres needle. When a Veres needle is inserted into the abdomen, the surgeon usually has to cross three main planes to reach the target, the abdominal cavity. Therefore, the three events to be heard are as follows:
Event 1: Tissue-tissue junction 1 (first fascia) puncture;
Event 2: Tissue-tissue boundary 2 (second fascia) puncture;
Event 3: Puncture of the target structure (peritoneum).

For other needle puncture tasks, fewer or more main planes may need to be traversed; the inventive method can be easily adapted to this.

The gray blocks represent active processing blocks for the given specific application with the steps 5a1, 5a2, 5a3, 5b, 5c, 5d2_{I}, 5d2_{II}, 5d2_{III}, 5d3 and forwarding to the output device 6. The output device 6 can be provided in such a way that it generates a specific optical, acoustic or haptic output for each event so that the user is informed of the progress of the puncture at all times. The naming of the process steps refers to FIG. 14.

FIG. 17 shows the flowchart for the specific processing for the automatic detection of laparoscopic instruments, such as in the configurations in FIGS. 11, 12 and 13.

A preferred computer-implemented method for automatic detection of laparoscopic instruments comprises the following steps:
(0) Providing a digitized vibroacoustic signal;
(I) Signal pre-processing of the known vibroacoustic signal (5a), comprising the process steps:
   i. Removing continuous components (DC) from the VA signal (5a1);
   ii. Background noise reduction (5a2);
   iii. Signal enhancement (5a3);
(II) Identification of segments/regions of interest (5b); and
(III) Classification (5e) of the laparoscopic tool, comprising the process steps:
   i. Spectral band decomposition (5e2);
   ii. Identification of spectrum-based indicators (5e4_{I});
   iii. Feeding the data into a deep learning algorithm (5e9); and
   iv. Classification using a machine learning model (5e10);

Optionally, the classification is stored in the output device (6) in a step (IV).

The numbering of the process steps refers to FIG. 14.

When a laparoscopic tool is inserted through the trocar, the generated interaction (inner part of the trocar with the instrument) results in a VA signal that can be used to classify the type of laparoscopic tool used in the surgical process. Since the signature of the VA signal can be very complex, feature extraction after steps 5a1, 5a2, 5a3, 5b, 5c, 5e2, 5e4_{I} is further performed using deep learning methods 5e9.

The features are fed into a trained machine learning model 5e10 that identifies the instrument. These informations can be stored for later use in the output device 6 or used to generate automatic operation reports. The naming of the process steps refers to FIG. 14.

FIG. 18 shows the flowchart for the specific data processing of an audification during laparoscopic procedures according to FIG. 11.

A preferred computer-implemented method for direct audification during laparoscopic procedures comprises the following steps:
(0) Providing a digitized vibroacoustic signal;
(I) Signal pre-processing of the known vibroacoustic signal (5a), comprising the process steps:
   i. Removing continuous components (DC) from the VA signal (5a1);
   ii. Background noise reduction (5a2);
   iii. Signal enhancement (5a3);
(II) Identification of segments/regions of interest (5b); and
(III) Direct audification (5d1), comprising the process steps of:
   i. Filtering the signal (5d1_{I});
   ii. Adjusting the volume (5d1_{II});
   iii. Processing the dynamics (5d1_{III});
   iv. Creation of effects (5d1_{IV});
(IV) Transmission (5d3) to the output device (6).

The numbering of the process steps refers to FIG. 14.

The main goal of laparoscopic audification is to add transparency to the soundless video endoscope by highlighting and auditing each tool-tissue interaction of the laparoscopic tools involved in a particular intervention. This can be used in real time to improve surgical perception during a real operation or for surgeon training. A direct audition algorithm can be used where the interactions of each tool involved in the surgery are processed in parallel and where each tool can be played back through different speakers.

FIG. 19 shows the data processing flowchart of a specific application for real-time vibroacoustic monitoring during electrosurgical interventions to characterize different tissues or different tissue conditions.

Electrosurgery and electrocautery play an essential role in minimally invasive surgery, especially in laparoscopic and robot-assisted surgery. Electrical energy is used to heat tissue in order to perform surgical tasks such as cutting or coagulation to stop bleeding. These procedures are crucial for the precise treatment of tissue in minimally invasive surgery.

Despite their usefulness, electrosurgical procedures come with challenges, including the risk of burns and unintended tissue damage. There is a critical need for technologies that can monitor tissue changes in real time during electrosurgical procedures. This feedback is essential for surgeons to avoid the aforementioned complications. To meet this need, the invention provides non-invasive monitoring methods also in real time. The identification of tissue conditions or phases during heating is crucial to optimize the energy output of the medical devices and avoid adverse effects.

The present invention can be used for monitoring during this type of procedure. The vibroacoustic sensor module, which can be connected to the proximal end of electrosurgical instruments (most are similar in appearance to standard laparoscopic instruments), enables real-time detection of vibroacoustic waves during the procedure. As the tissue characteristics change with the tissue effects produced by the electrical heating, these changes can be captured and recognized by the vibroacoustic signals. The invention can interpret these signals and allows the surgeon to identify and track tissue changes, even in real time. By converting these signals into information to monitor the effects of heating on the tissue, surgeons gain insight to adjust the energy output during electrosurgical device activation and the timing of electrosurgical device exposure.

In practical use, the invention serves as an auxiliary device that is connected to electrosurgical instruments, as shown in FIG. 11. During the electrosurgical procedure, the combination of the sensor unit and processing unit detects changes in tissue characteristics and enables real-time identification and tracking. Surgeons receive immediate feedback on tissue changes, allowing them to make informed adjustments during the procedure. This capability makes the invention a valuable tool for increasing precision, reducing complications and improving patient outcomes in electrosurgery.

Many of the electrosurgical tools have the same characteristics as standard laparoscopic tools. Therefore, the embodiment shown in FIG. 11 is also suitable for this application, as the sensor module can be connected to the electrosurgical instrument via an adapter in the shaft or indirectly via the Luer lock of the trocar. The vibroacoustic signal is captured by the sensor module during the heating process. The signal is transmitted wirelessly or wired to a main unit, which processes the signal and classifies the vibroacoustic patterns according to the heating level of the tissue.

FIG. 19 shows in gray the blocks used for the method according to the invention in connection with electrosurgery from the general processing chart of FIG. 14.

The basic idea of the invention to capture vibroacoustic signals and break them down into different features is also applied accordingly in this context. The features can then be linked to specific tissue properties.

Accordingly, the method for feature extraction and classification in electrosurgery comprises the following steps:
(0) Providing a digitized vibroacoustic signal;
(I) Signal preprocessing of the known vibroacoustic signal (5a), comprising the process steps of
   i. Removing continuous components (DC) from the VA signal (5a1);
   ii. Background noise reduction (5a2);
   iii. Signal enhancement (5a3);
(II) Classification of the vibroacoustic signals generated during electrosurgery into one of the specified classes, in particular a tissue heat level (5e), comprising the process steps of
   i. Identification of transient and steady-state dynamics (5e1);
   ii. Spectral band decomposition (5e2);
   iii. Frequency and time-frequency domain indicators (5e4), namely:
      1. Identification of spectrum-based indicators (5e4_{I});
      2. Indicators based on instantaneous dominant frequency (5e4_{II}); and
   iv. Spectral domain features (5e7), namely:
      1. Frequency-spectrum-event features (5e7_{I});
      2. Time-frequency spectrum event features (5e7_{II});
      3. Statistical features of the instantaneous dominant frequency (5e7_{III});
      4. Dynamic features of instantaneous dominant frequency (5e7_{IV});
   v. Classification by a machine learning model (5e10).

Optionally, in a step (IV), the classification is output by an output device 6 in haptic and/or acoustic and/or optical form.

The numbering of the process steps refers to FIG. 14.

During data processing (step 5e10), the vibroacoustic signal resulting from the various tissue heat levels is categorized into the following classes in particular:
Class 1: Hyperthermia (from 40°): initial tissue damage, edema formation, depending on the duration of application, the tissue may recover or die (devitalization).
Class 2: Devitalization (destruction) of the cells, shrinkage of the connective tissue due to denaturation (from 60°).
Class 3: Evaporation of the tissue fluid, depending on the speed of evaporation (around 100°).
Class 4: Carbonization (from approx. 150°).

FIG. 20 shows the flowchart for data processing for a specific application for real-time vibroacoustic monitoring for precise bone drilling or screwing (hereinafter “drilling” and “screwing” are simplified as “drilling” or “bone drilling”) in surgical interventions.

Bone drilling is required in various surgical disciplines such as orthopaedic surgery, ear surgery, maxillofacial surgery and neurosurgery. In these interventions, the challenge is that bone drilling is manual and surgeons often have difficulty removing the right amount of material or reaching the desired depth. The inertia of the drilling force and physiological shocks can lead to unintended damage, for example, a drill may be advanced further than intended during a breakthrough. The current dependence of successful intervention on the surgeon's experience and intuition highlights the need for a non-invasive and objective monitoring solution.

Manual bone drilling is a challenge for the surgeon as it is difficult to precisely control the intervention and especially the drilling. Electronic monitoring of bone drilling is also difficult as there is no immediate feedback, especially when breakthroughs occur. Current monitoring methods, such as force measurement and acoustic emission, have their own problems. Force sensing is slow to respond to rapid changes during drilling and has a limited range. Acoustic emission is sensitive to the noise generated by the drilling process itself.

The invention addresses the unmet clinical need for non-invasive monitoring during bone drilling. In contrast to conventional methods, the invention captures vibroacoustic waves whose dynamics are directly related to density changes, including bone fractures. This approach enables real-time processing of the resulting vibroacoustic waves to monitor the bone drilling process. The properties of the vibroacoustic waves captured by the system change their behavior when the bone density changes. In this way, it is possible to characterize the density changes in real time by extracting features from the vibroacoustic signal.

For this purpose, the sensor module can be attached to the proximal end of the drill via a standard connection such as a drill chuck or a specially designed adapter, as shown in FIG. 11A. The vibroacoustic signal is sensed by the sensor module and transmitted wirelessly or wired to a main unit that processes the signal to provide real-time feedback when the drill transitions from one type of bone density to another type of bone density or when it breaks the bone. The signal processing path mainly includes pre-processing, spectral and envelope-based feature extraction, and finally a trained machine learning model that recognizes the moment when the bone density changes and bone breakage occurs. The moment the algorithm recognizes these changes, it provides feedback to the user.

Accordingly, the process for precise bone drilling in surgical procedures includes the following steps:
(0) Providing a digitized vibroacoustic signal;
(I) Signal pre-processing the known vibroacoustic signal (5a), comprising the process steps of
   i. Removing continuous components (DC) from the VA signal (5a1);
   ii. Background noise reduction (5a2);
   iii. Signal enhancement (5a3);
(II) Identification of segments/regions of interest (5b);
(III) Classification of the vibroacoustic signals generated during drilling into one of the specified classes, in particular a bone layer class (5e, see FIG. 11A), comprising the process steps of
   i. Identification of transient and stationary dynamics (5e1);
   ii. Spectral band decomposition (5e2);
   iii. Indicators in the frequency and time-frequency domain (5e4), namely:
      1. Identification of spectrum-based indicators (5e4_{I});
      2. Indicators based on the instantaneous dominant frequency (5e4_{II}); and
   iv. Envelope-based indicators (5e5);
   v. Spectral domain features (5e7), namely:
      1. Frequency-spectrum-event features (5e7_{I});
      2. Time-frequency spectrum event features (5e7_{II});
      3. Statistical features of the instantaneous dominant frequency (5e7_{III});
      4. Dynamic characteristics of the instantaneous dominant frequency (5e7_{IV});
   vi. Spectral envelope features (5e8_{I});
   vii. Classification by a machine learning model (5e10).

Optionally, in a step (IV), the classification is output by an output device 6 in haptic and/or acoustic and/or optical form and indicates to the user, for example, a change in bone density.

The numbering of the process steps refers to FIG. 14.

FIG. 21 shows the flowchart of the flow chart of a specific application for the detection and prevention of damage to pulsating structures in laparoscopic and robotic-assisted surgery.

In robotic-assisted and laparoscopic interventions, accurate detection of pulsatile structures, such as blood vessels, is critical to prevent accidental damage. Currently, the surgeon's perception of these structures relies on visual cues and individual experience. The challenge in capturing such structures lies in situations where pulsatile structures are not immediately recognizable or visible during surgery. Thus, there is a clinical need for technologies that can detect these pulsatile structures in real time to increase the safety of these procedures by providing real-time feedback to the surgeon about the proximity of a pulsatile structure, thereby reducing the risk of inadvertent damage. Alternatively, information about the presence of a pulsating structure can be relayed to a robotic system if the surgeon does not notice a pulsating structure nearby. This inventive approach ensures that even if the surgeon initiates a potentially harmful action, the robotic system can prevent it from being performed.

To solve this problem, the invention can automatically and in real time detect the presence of these pulsating structures and provide direct feedback to the surgeon and/or the robot.

When the tip of a laparoscopic or robotic instrument comes into direct contact with a vessel or the surrounding connective or covering tissue, the vessel pulsates and causes movement in the surrounding tissue as well. This interaction generates vibrations at the tip of the instrument, which propagate through the instrument and are captured by the sensor module. This vibroacoustic signal is then transmitted either wirelessly or via a wire to a central processing unit. This unit processes the signal and makes it possible to detect the presence of a pulsating structure in the vicinity and inform the user of this event.

Corresponding embodiments of the invention can also be found in FIG. 11 and 11B.

The data processing, as shown in the flowchart, primarily comprises a pre-processing stage to reduce background noise and enhance the periodic interactions associated with the pulsation. Subsequently, the pulsations are identified and their intensity is translated into a periodic acoustic feedback with background noise that correlates with both the intensity and the frequency of the pulsation. The closer the instrument is to the pulsating structure, the higher the intensity of the periodic acoustic feedback. In this way, the user can judge the proximity to a pulsating structure—no acoustic feedback means that there is no pulsating structure nearby.

The gray blocks represent active processing blocks for the given specific application with steps 5a1, 5a2, 5a3, 5b, 5c, 5d2_{I}, 5d2_{II}, 5d2_{III}, 5d3 and forwarding to the output device 6. The output device 6 may be provided to generate a specific output in visual, audible or haptic form for each event so that the user is aware of the proximity to a pulsating object at all times.

A preferred computer-implemented method for capturing and preventing damage to pulsating structures in laparoscopic and robotic surgery comprises the following steps:
(0) Providing a digitized vibroacoustic signal;
(I) Signal pre-processing the known vibroacoustic signal (5a), comprising the process steps of
   i. Removing continuous components (DC) from the VA signal (5a1);
   ii. Background noise reduction (5a2);
   iii. Signal enhancement (5a3);
(II) Identification of segments/regions of interest (5b); and
(III) Indirect audification (5d2), comprising the process steps of
   i. Feature extraction (5d2_{I});
   ii. Mapping of the extracted features (5d2_{II});
   iii. Generation of sound characteristics (5d2_{III}); and
(IV) Transfer (5d3) to the output device (6).

The numbering of the process steps refers to FIG. 14.

FIG. 22 shows the flowchart for data processing for a specific application for real-time detection and classification of collisions in robot-assisted operations using the technology according to the invention.

In robotic surgery, the lack of haptic feedback can lead to unrecognized collisions, causing delays and potential damage. Cases such as robot arms blocking each other or collisions between laparoscopic instruments often go unnoticed by the surgeon and prolong the operation time. It becomes even more critical when the robot's tool collides with soft tissue, which can lead to damage that the surgeon does not immediately recognize. The technology according to the invention provides a solution.

The sensor module detects vibroacoustic signals generated by various collisions—be it collisions of instruments, blockages of the robot arm or contact with soft tissue. The different characteristics of these vibroacoustic signals make it possible to identify and classify collisions in real time and provide surgeons with immediate feedback, thus contributing to the safety and efficiency of robot-assisted operations. Details can also be found in FIG. 11B.

Accordingly, the feature extraction and classification method for real-time detection and classification of collisions in robotic surgeries using the technology of the invention comprises the following steps:
(0) Providing a digitized vibroacoustic signal;
(I) Signal pre-processing the known vibroacoustic signal (5a), comprising the process steps of
   i. Removing continuous components (DC) from the VA signal (5a1);
   ii. Background noise reduction (5a2);
   iii. Signal enhancement (5a3);
(II) Identification of segments/regions of interest (5b); and
(III) Classification of the vibroacoustic signals generated during robotic operations into one of the specified classes, in particular a collision class:
   i. Identification of transient and steady-state dynamics (5e1);
   ii. Spectral band decomposition (5e2);
   iii. Calculation of time domain indicators (5e3), namely:
      1. Statistical indicators (5e3_{I});
      2. Morphological indicators (5e3_{II});
      3. Indicators based on sudden changes (5e3_{III});
   iv. Time domain characteristics (5e6), namely:
      1. Statistical indicators (5e6_{I});
      2. Morphological indicators (5e6_{II});
      3. Indicators based on sudden changes (5e6_{III});
   v. Indicators in the frequency and time-frequency domain (5e4), namely:
      1. Identification of spectrum-based indicators (5e4_{I});
      2. Indicators based on instantaneous dominant frequency (5e4_{II}); and
   vi. Spectral domain features (5e7), namely:
      1. Frequency-spectrum-event features (5e7_{I});
      2. Time-frequency spectrum event features (5e7_{II});
      3. Statistical features of the instantaneous dominant frequency (5e7_{III});
      4. Dynamic features of instantaneous dominant frequency (5e7_{IV});
   vii. Classification by a machine learning model (5e10).

Optionally, in a step (IV), the classification is output by an output device 6 in haptic and/or acoustic and/or optical form.

the numbering of the process steps refers to FIG. 14.

One or more sensor modules are connected to the proximal end of robot arms whose instruments require collision monitoring. The sensor module detects vibroacoustic signals and transmits them wirelessly or via wire to a main unit for signal processing. The signal processing path shown in FIG. 22 includes, in particular, a pre-processing stage to reduce background noise and improve interaction. Subsequently, possible collision events (region of interest) are identified and features are extracted from each event to determine whether it is a collision. If a collision is confirmed, the machine learning model trained for this purpose classifies the type of collision. The classification includes:
Class 1: No collision;
Class 2: Collision between instrument and instrument;
Class 3: Collision with the robot arm;
Class 4: Collision between instrument and soft tissue.

### Reference Sign List

1 Interactive tissue
1a Surface of the tissue
1b Target/anatomical structure in the tissue
1c Target/anatomical structure, which is covered by tissue
1d Target/anatomical structure in the cavity under the tissue
1e Tissue-tissue boundary
1f Edge of the tissue cavity
2 Medical instrument (2a: trocar; 2b: laparoscope; 2c: drill, bone screw)
3 Adapter element
4 Signal acquisition device
4a Coupling element
4b Mechanical amplification mechanism
4c Acoustic sensor or vibration sensor
4d Analog signal pre-processing
4e Analog-to-digital conversion
4f Controller
4g Communication unit
5 Data processing/signal processing device
6 Output device
6a Loudspeaker
6b Memory/database
6c VR/AR device
6d Screen for visualization
6e Haptic device
6f Report
7 Body
8 Endoscopic camera system
8a Camera
8b Endoscope
8c Field of view (FOV)
8d Line of sight (LOS)
9 Robotic instrument
10 Surgical robot/robotic arm
11 Surgical drill machine
12 Bone
12a Bone layer with the density a
12b Bone layer with the density b
12c Bone layer with the density c
12d Last bone layer before drilling through
13 Collision

1. Device for connection to a medical instrument for capturing vibroacoustic signals, wherein the device comprises a signal capturing device (4) with a coupling element (4a) for connecting a medical instrument (2), at least one sensor (4c) for capturing vibroacoustic signals, an analog-to-digital conversion (4e) for converting the analog signals captured by the at least one sensor into digital data, a controller (4f) and a communication unit (4g) for data exchange.

2. Device according to claim 1, wherein the coupling element (4a) connects the medical instrument (2) and the at least one sensor (4c) in a sound-transmitting manner, wherein the coupling element (4a) is suitable for transmitting vibroacoustic signals from the instrument (2) to the at least one sensor (4c) with as little interference and loss as possible, and wherein the at least one sensor (4c) is connected directly to the coupling element (4a), in particular in a force-locking, form-locking or friction-locking manner.

3. Device according to claim 1, wherein the signal acquisition device (4) comprises a mechanical amplification mechanism (4b) with a membrane and a sound chamber, wherein the coupling element (4a) is suitable for transmitting vibroacoustic signals from the instrument (2) to the membrane with as little interference and loss as possible and is connected to the membrane not permanently but at least in a force-locking or friction-locking manner, i.e. in an effect-transmitting manner, wherein the membrane transmits the vibroacoustic signals into a sound chamber adjoining the membrane, wherein the vibroacoustic signals have an impulse component which acts in the axial direction, i.e. perpendicular to the membrane, and which leads to an excitation or deflection of the membrane, and has an additional bending moment at the coupling point of the coupling element (4a) with the membrane, wherein both the axial impulse component and the bending moment generate a deflection of the membrane and thus a sound wave or a pressure wave in the sound chamber adjoining the membrane, and wherein the at least one sensor (4c) is provided at the sound chamber and preferably delimits it.

4. A computer-implemented method for processing and evaluating vibroacoustic signals, comprising the following steps:
(0) Providing an existing digitized vibroacoustic signal;
(I) Signal pre-processing of the vibroacoustic signal (5a); and
(II) Identification of segments/regions of interest (5b); and
(III) Audification (5d); and/or (5c)
(IV) Classification of the segments/regions of interest (5e).

5. Computer-implemented method for processing and evaluating vibroacoustic signals according to claim 4 for feature extraction and classification of signals generated during palpation, comprising the following steps:
(0) Providing a digitized vibroacoustic signal;
(1) Signal pre-processing the known vibroacoustic signal (5a), comprising the process steps of:
i. Removing continuous components (DC) from the VA signal (5a1);
ii. Background noise reduction (5a2);
iii. Signal enhancement (5a3);
(II) Identification of segments/regions of interest (5b); and
(III) Classification of the vibroacoustic signals generated during palpation into one of the specified classes, in particular a softness/hardness class (5e), comprising the process steps:
i. Identification of transient and steady-state dynamics (5e1);
ii. Spectral band decomposition (5e2);
iii. Calculation of time domain indicators (5e3), namely:
1. Statistical indicators (5e3_{I});
2. Morphological indicators (5e3_{II});
3. Indicators based on sudden changes (5e3_{III});
iv. Time domain characteristics (5e6), namely:
1. Statistical indicators (5e6_{I});
2. Morphological indicators (5e6_{II});
3. Indicators based on sudden changes (5e6_{III});
v. Indicators in the frequency domain and time-frequency domain (5e4), namely:
1. Identification of spectrum-based indicators (5e4_{I});
2. Indicators on the basis of the instantaneous dominant frequency (5e4_{II}); and
vi. Spectral domain features (5e7), namely:
1. Time-frequency spectrum event features (5e7_{II});
2. Statistical features of instantaneous dominant frequency (5e7_{III});
vii. Classification by a machine learning model (5e10).

6. Computer-implemented method for processing and evaluating vibroacoustic signals according to claim 4 for indirect audification for indirect testing of a needle puncture, comprising the following steps:
(0) Providing a digitized vibroacoustic signal;
(I) Signal pre-processing the known vibroacoustic signal (5a), comprising the process steps of:
i. Removing continuous components (DC) from the VA signal (5a1);
ii. Background noise reduction (5a2);
iii. Signal enhancement (5a3);
(II) Identification of segments/regions of interest (5b); and
(III) Indirect audification (5d2), comprising the process steps:
i. Feature extraction (5d2_{I});
ii. Mapping of the extracted features (5d2_{II});
iii. Generation of sound features (5d2_{III}); and
(IV) Transmission (5d3) to the output device (6).

7. Computer-implemented method for processing and evaluating vibroacoustic signals according to claim 4 for automatically recognizing laparoscopic instruments, comprising the following steps:
(0) Providing a digitized vibroacoustic signal;
(I) Signal pre-processing the known vibroacoustic signal (5a), comprising the process steps of:
i. Removing continuous components (DC) from the VA signal (5a1);
ii. Background noise reduction (5a2);
iii. Signal enhancement (5a3);
(II) Identification of segments/regions of interest (5b); and
(III) Classification (5e) of the laparoscopic tool, comprising the process steps:
i. Spectral band decomposition (5e2);
ii. Identification of spectrum-based indicators (5e4_{I});
iii. Feeding the data into a deep learning algorithm (5e9); and
iv. Classification by a machine learning model (5e10).

8. Computer-implemented method for processing and evaluating vibroacoustic signals according to claim 4 for direct audification during laparoscopic procedures, comprising the following steps:
(0) Providing a digitized vibroacoustic signal;
(I) Signal pre-processing the known vibroacoustic signal (5a), comprising the process steps of:
i. Removing continuous components (DC) from the VA signal (5a1);
ii. Background noise reduction (5a2);
iii. Signal enhancement (5a3);
(II) Identification of segments/regions of interest (5b); and
(III) Direct audification (5d1), comprising the process steps of:
i. Filtering the signal (5d1_{I});
ii. Adjusting the volume (5d1_{II});
iii. Editing the dynamics (5d1_{III});
iv. Generation of effects (5d1_{IV});
(IV) Transmission (5d3) to the output device (6).

9. A method according to claim 4, wherein the vibroacoustic signals are provided by a device for connection to a medical instrument for capturing vibroacoustic signals, wherein the device comprises a signal capturing device (4) with a coupling element (4a) for connecting a medical instrument (2), at least one sensor (4c) for capturing vibroacoustic signals, an analog-to-digital conversion (4e) for converting the analog signals captured by the at least one sensor into digital data, a controller (4f) and a communication unit (4g) for data exchange.

10. A device according claim 1, wherein the device comprises a data processing/signal processing device (5) provided to perform the computer-implemented method according to a computer-implemented method for processing and evaluating vibroacoustic signals, comprising the following steps:
(0) Providing an existing digitized vibroacoustic signal;
(I) Signal pre-processing of the vibroacoustic signal (5a); and
(II) Identification of segments/regions of interest (5b); and
(III) Audification (5d); and/or (5c)
(IV) £ Classification of the segments/regions of interest (5e).

11. The device according to claim 1, wherein in a further step (V), the output, further processing or storage (6) of the classified segments/regions takes place in an output device.

12. The computer-implemented method of claim 5, wherein in a step (IV) the classification is stored in the output device (6).

13. The computer-implemented method of claim 4, wherein in a further step (V) the output, further processing or storage (6) of the classified segments/regions takes place in an output device.

14. The computer-implemented method of claim 7, wherein in a step (IV) the output of the classification is provided by an output device (6) in haptic and/or acoustic and/or optical form.
